(19) United States
(12) Patent Application Publication
(10) Pub. No.: US 2023/0149683 A1
(43) Pub. Date: May 18, 2023

(54) Methods for Enhancing Transdermal and Intradermal Delivery of Glycosaminoglycans (GAGs)

(71) Applicant: B. G. Negev Technologies and Applications Itd., at Ben-Gurion university, Beer Sheva (IL)

(72) Inventors: Leah Shimonov, Beer Sheva (IL); Joseph Kost, Beer Sheva (IL); Tamar Traitel, Beer Sheva (IL); Riki Goldbart, Beer Sheva (IL)

(21) Appl. No.: 17/904,901

(22) PCT Filed: Feb. 24, 2021
(86) PCT No.: PCT/IB2021/051558

§ 371 Date: Aug. 24, 2022

Related U.S. Application Data
(60) Provisional application No. 62/980,635, filed on Feb. 24, 2020.

(51) Int. Cl. A61M 37/00; A61Q 19/08; A61K 8/73

(52) U.S. Cl. CPC A61M 37/0092; A61Q 19/08; A61K 8/735; A61K 8/732; A61K 2800/82; A61M 2210/04

(57) ABSTRACT

Methods for transdermal delivery of glycosaminoglycans (GAGs) are disclosed, comprising applying ultrasound to the skin followed by topical administration of one or more GAGs optionally complexed with a polysaccharide carrier. When GAG is hyaluronic acid complexed with a modified starch, a transdermal delivery method is provided for facilitating delivery of high molecular weight hyaluronic acid into deep layers of the epidermis and to the dermis in a non-invasive, convenient and painless way. This transdermal delivery method may be applied in treatment of skin aging phenomena associated with collagen and/or hyaluronic acid depletion or loss.

### FIELD OF THE INVENTION

The present disclosure relates to methods of enhancing the transdermal delivery of glycosaminoglycans (GAGs), more specifically, but not exclusively, to ultrasound enhanced transdermal delivery of hyaluronic acid (HA).

### BACKGROUND

Human skin aging is a complex biological process, mediated by a combination of two independent factors. The first process is intrinsic or innate aging, affected by hormonal changes that occur with aging, such as diminution of estrogen, androgen and progesterone, which is associated with menopause and andropause (male menopause). Deficiency in these hormones results in collagen degradation, dryness, loss of elasticity, skin atrophy and wrinkling of the skin. The second process is extrinsic aging, which is the result of exposure to external factors, mainly ultraviolet (UV) irradiation. The key molecule involved in skin moisture and improvement of collagen production is hyaluronan or hyaluronic acid (HA), a glycosaminoglycan (GAG; long unbranched polysaccharides consisting of a repeating disaccharide unit) that forms the main component of the extracellular matrix (ECM). Youthful skin is hydrated because it contains large amounts of HA in the dermis. However, as we age, the amount of HA in the skin decreases and by the time we become adults, this amount decreases to five percent of baseline. The combination of fibers and ECM provides to the skin viscoelastic properties and consequent strength and resilience, but with aging, disorganization and degradation of dermic fibers and HA occur resulting in reduction of HA's ability to impart to the skin elasticity, density and resistance.

The stratum corneum (SC), the outer layer of the skin, provides mechanical protection to the skin and is a barrier to water loss and permeation of substances from the environment. Particularly, the SC prevents efficient penetration of large molecules (>500 Da), thus, effective utilization of topically administered HA having a large molecular size is limited due to skin permeability and, practically, HA topically applied does not penetrate through the epidermis all the way down to the dermis. Therefore, when preventing or treating skin aging processes is desired, HA is usually delivered to deeper layers of the skin by injections. The main disadvantages of such invasive treatments can range from mild symptoms such as localized pain or swelling to more serious problems such as serious injury due to penetration into blood vessels in the skin.

There is yet an unmet need for a non-invasive means for transdermal delivery of high molecular weight HA.

### SUMMARY

When topically applied onto the skin, HA lacking the ability to permeate the stratum corneum layer, remains on the skin's surface and functions as a skin-surface moisturizer. Delivery of high molecular weight HA into deeper skin layers is highly desired because it reaches more tissue and has longer duration. Currently, transdermal delivery of high molecular weight HA for a wide range of applications is performed by intradermal (ID) injection of HA, i.e., injections delivered into the dermis.

The ability of low frequency ultrasound application to enhance biological membranes permeability, and mostly skin permeability, for large molecules, has been extensively studied by the present inventors, and a non-invasive delivery system for transdermal delivery of high molecular weight glycosaminoglycans (GAGs) such as HA has been envisaged, which may find use, inter alia, in skin aging treatment. The present inventors have successfully practiced delivery of HA into the epidermis and dermis by application of ultrasound in combination with utilization of chemically modified starch as the HA carrier.

Disclosed herein is a platform or system that combines ultrasound application followed by topical administration of HA (as well as other GAGs) complexed with a polysaccharide carrier. This platform provides a convenient, painless treatment for wrinkles filling, delaying aging process, reducing aging indicators associated with loss of mechanical properties, and restoring skin moisture for skin smoothing. The disclosed platform may find further use in multiple therapeutic treatments which currently involve HA injection such as treatment of knee pain caused by osteoarthritis.

In one aspect, the present disclosure relates to a non-invasive method for preventing or treating skin aging processes in a subject in need thereof, the method comprising the steps of:

(a) applying ultrasound treatment to a skin surface of the subject for a period of from about 5 sec to about 5 min;

(b) topically administrating to the ultrasound-treated skin surface at least one of: free hyaluronic acid (HA) or HA complexed with a polysaccharide (HA-polysaccharide complex); and

(c) optionally, repeating at least one of step (a) or (b) at least one more time, thereby non-invasively preventing or treating skin aging processes in the subject.

The disclosed method is suitable for transdermal delivery of HA of any molecular weight (MW) and, particularly, for delivery of high molecular weight (HMW) HA (>1000 kDa).

A disclosed method is useful in maintaining skin hydration, restoration or improvement of collagen production, delaying aging process such as wrinkling, or reducing aging indicators associated with loss of mechanical properties such as skin atrophy, or loss of skin elasticity.

In another aspect, the present disclosure relates to a method for transdermal delivery of one or more glycosaminoglycans (GAGs) in a subject in need thereof, the method comprising the steps of:

(a) optionally, forming a complex comprising one or more GAGs and at least one polysaccharide (GAG-polysaccharide complex);

(b) applying ultrasound treatment to a skin surface of the subject for a period of from about 5 sec to about 5 min;

(c) topically administrating one or more GAGs and/or one or more GAG-polysaccharide complexes to the ultrasound-treated skin surface;

(d) optionally, applying a further ultrasound treatment to the skin surface for a period of from about 5 sec to about 5 min; and

(e) optionally, topically administrating to the ultrasound-treated skin surface, a further amount of one or more GAGs and/or one or more GAG-polysaccharide complexes, thereby transdermally delivering one or more GAGs in the subject.

In some embodiments, step (a) is not applied.

In some embodiments, step (a) is applied, and at least one GAG-polysaccharide complex is topically administered in step (c) and/or step (e).

In some embodiments, at least one of step (d) or step (e) is not applied. In some embodiments, at least one of step (d) or step (e) is applied 1, 2, 3 or more times.

The GAGs delivered transdermally by a disclosed method may be, for example, hyaluronic acid, heparin, heparan sulfate, chondroitin sulfate, dermatan sulfate, or keratan sulfate having a MW of from 300 kDa to 8000 KDa, for examples, from 500 kDa to 3000 kDa, or of from 300 kDa to 800 KDa.

The polysaccharide employed in a contemplated method may be at least one of starch, chitosan, pectin, cellulose, dextran, or galactan, optionally chemically modified, for example, modification by substitution with one or more positively charged chemical moieties, such as, but not limited to, a quaternary amine group.

In some embodiments starch substituted with quaternary amine groups (Q-starch) is utilized as a carrier for HA.

In a further aspect, the present disclosure relates to a complex of hyaluronic acid and a chemically modified starch, wherein starch has been modified by substitution with one or more quaternary amine groups such as (CH₃)₃—N⁺—.

In some embodiments, a disclosed complex features a molar ratio of positively charged chemical moieties of the modified starch and negatively charged carboxyl groups of hyaluronic acid (N/O molar ratio) which is in a range of from about 0.20 to about 3.00, for example, from about 0.25 to about 1.5.

In yet a further aspect, the present disclosure relates to a composition comprising a complex of hyaluronic acid and a chemically modified starch as defined herein, and at least one physiologically acceptable excipient. The contemplated composition may be a cosmetic composition or a therapeutic composition (i.e., a medicament).

In still a further aspect, the present disclosure relates to a kit comprising: (a) at least one complex of hyaluronic acid and a chemically modified starch as defined herein or a composition comprising same; (b) means for applying ultrasound treatment; and (c) optionally, instructions and means for administration of the complexed hyaluronic acid and/or the composition to a subject.

Any of the complexes, compositions and/or kits contemplated herein may find use in enhancing non-invasive transdermal delivery of hyaluronic acid, preferably HMW HA, for the purpose of, e.g., anti-aging treatment.

### BRIEF DESCRIPTION OF THE FIGURES

Some embodiments of the present disclosure are herein described, by way of example only, with reference to the accompanying drawings. With specific reference now to the drawings in detail, it is stressed that the particulars shown are by way of example and for purposes of illustrative discussion of embodiments described herein. In this regard, the description taken with the drawings makes apparent to those skilled in the art how embodiments of the disclosure may be practiced.

In the drawings:

FIGS. 1A-1B are graphs showing the size distribution of complexes of quaternary starch (Q-starch) and hyaluronic acid (HA) (Q-starch-HA complexes) (1A), and of free Q-starch and HA (1B) obtained using dynamic light scattering (DLS). The Q-starch-HA complexes feature increasing ratios between positively charged amine groups on Q-starch (N), and negatively charged carboxylic groups on HA backbone (O) (N/O ratios);

FIG. 2 is a graph showing the size distribution (average diameter) of free Q-starch, free HA and of Q-starch-HA complexes having N/O 0.25, measured using a NanoSight system;

FIG. 3 is a bar graph showing the mean ζ-potential (a function of the surface charge of a particle) of free HA, Q-starch, and Q-starch-HA complexes featuring increasing N/O ratios;

FIGS. 4A-4G are exemplary Cryo-TEM images of free (non-complexed) Q-starch (4A), free (non-complexed) HA (4B) and freshly prepared Q-starch-HA complexes at N/O molar ratios ranging from 0.25 to 3 (4C-4G);

FIGS. 5A-5B are bright field confocal images of exemplary porcine ear skin cross-sections following 24 hr incubation with 0.3% (w/v) HA labeled with Hylite™ Fluor 647 dye (HA^{Hylite Fluor 647}) in absence of ultrasound (US) pretreatment (5A) or following US application for 5 min (5B). The stratum corneum (SC), epidermis and dermis layers are indicated (Bar: 20 μm). Fluorescence intensity of the HA^{Hylite Fluor 647} as a function of distance from SC, down to a depth of 200 μm, calculated for pixels in an exemplary indicated rectangular cross-layers section by Image j, is shown for each cross section. Vertical dashed lines represent a separation between the layers. The horizontal dashed line indicates the skin's autofluorescence at the wavelength of the labeled HA;

FIGS. 6A-6D are confocal images of an exemplary porcine ear skin cross sections, histologically stained following 24 hr incubation with a labeled Q-starch/HA complex (Q-starch-HA^{Hylite Fluor 647}) featuring N/O molar ratio of 0.25. The Skin samples were either not pre-treated with ultrasound application prior to topical application of the labeled Q-starch/HA complex (6A, 6B) or treated with US application for 5 minutes before complex application (6C, 6D). FIGS. 6A and 6C are confocal images demonstrating intact nucleated cells in skin layers beneath the stratum corneum (SC) (nuclei staining with 4′,6-diamidino-2-phenylindole (DAPI); FIGS. 6B and 6D are bright field confocal images. The SC, epidermis and dermis layers are indicated (Bar: 20 μm). Fluorescence intensity of the Q-starch-HA^{Hylite Fluor 647} as a function of distance from SC, down to a depth of 350 μm, calculated for pixels in an exemplary indicated rectangular cross-layers section by Image j, is shown for each cross section. Vertical dashed lines represent a separation between the layers. The horizontal dashed line indicates the skin's autofluorescence at the wavelength of the labeled HA;

FIG. 7 is a bar graph showing fluorescence intensities measured at labeled HA (HA^{Hylite Fluor 647}) wavelength in three layers of porcine ear skin: SC (0-20 μm), epidermis (20-100 μm) and dermis (100-2000 μm). Three groups of skin samples were observed: (i) skin samples topically applied with labeled Q-Sratch-HA complex (Q-starch-HA^{Hylite Fluor 647}) for 24 hours; (ii) skin samples treated with ultrasound for 5 min and then topically applied with Q-starch-HA^{Hylite Fluor 647} for 24 hours; and (iii) control group—skin samples not treated with neither ultrasound nor labeled complex. This groups serves for autofluorescence measurements. The fluorescence intensity was calculated by Image j based on data recorded from confocal scanning (three repetitions±SEM); and

FIGS. 8A-8D are confocal images of an exemplary porcine ear skin cross sections, histologically stained following 24 hr incubation with a labeled Q-starch/HA having N/O molar ratio of 0.25, wherein Q-starch was labeled with 5-(4,6-dichlorotriazinyl) aminofluorescein (5-DTAF) (Q-starch^{5-DTAF}) and appears as bright green staining in images 8A and 8C, and HA was labeled with Hylite™ Fluor 647 (HA^{Hylite Fluor 647}) and appears as red staining in images 8B and 8D. Nuclei of intact cells beneath the SC are stained in blue (DAPI-staining). The Skin samples were either not pre-treated with ultrasound application prior to topical administration of the labeled complex Q-starch^{5-DTAF}-HA^{Hylite Fluor 647} (8A, 8B) or treated with US application for 5 minutes before complex application (8C, 8D). Bar: 20 μm.

### DETAILED DESCRIPTION

The present disclosure relates to non-invasive means for enhancing the transdermal delivery of glycosaminoglycans (GAGs), more specifically, but not exclusively, to the application of ultrasound for enhancing transdermal delivery of hyaluronic acid (HA).

In the context of the present disclosure, the term “transdermal delivery” is to be interpreted, in a broad sense, as including both (i) an administration means of delivering a substance by way of the skin, namely, by application thereof onto the skin (topically), e.g., in a solution, ointment, patch and the like, so as to facilitate absorbance thereof systemically; and (ii) delivery through the upper, external stratum corneum (SC) skin layer into deeper skin layers such as the epidermis and dermis, e.g., down to a depth of at least 350 μm beneath the SC. This latter mode of delivery is also referred to herein as “intradermal delivery” or “intra skin layers delivery”. Thus, in any one of the embodiments described herein, transdermal delivery may apply to systemic delivery of GAGs by the skin and/or delivery of GAGs in-between the skin layers.

The present disclosure is based on the discovery by the present inventors that skin permeability of HA can be enhanced by applying ultrasound treatment to the skin. The present disclosure is further based on the discovery by the present inventors that when HA is allowed to self-assemble with positively charged starch i.e., starch substituted with positively charged moieties such as quaternary ammonium groups), a HA-starch complex is formed that can be readily delivered transdermally following ultrasound application to the skin, and moreover, it is stable in deep layers of the skin tissue. Such a complex afforded higher intra-tissue stability of HA as compared to the free acidic glycosaminoglycan and, therefore, longer HA retention time in deeper layers of the skin such as epidermis and dermis.

The present inventors have envisaged therapeutic and cosmetic hyaluronic acid-based treatment modalities, wherein HA is non-invasively instilled transdermally by way of utilizing application of ultrasound and HA assembly with quaternary starch (Q-starch). For example, the present inventors have envisaged combining ultrasound application and Q-starch-HA complexes in skin aging treatment modalities such as treatment of wrinkles, delaying aging process, reducing aging indicators associated with loss of mechanical properties, restoring skin moisture and smoothing the skin, wherein the combination of ultrasound application and HA complexing provides a convenient, non-invasive and painless means to deliver HA to target skin layers such as the dermis and, moreover, lessens the need for frequent transdermal administrations of HA.

The Examples disclosed herein describe the successful insertion of high molecular weight hyaluronic acid (1500 KDa) mainly to upper skin layer by applying ultrasound treatment to skin prior to HA application. A more significant penetration of HA to even deeper layers of the skin (epidermis, dermis) occurred when HA was complexed with a Q-starch before being applied to the skin. Complexes of Q-starch and HA (Q-starch-HA) were obtained for several molar ratios between positively charged amine groups on the Q-starch and negatively charged carboxylic groups on HA (herein referred to as N/O molar ratios or, simply, N/O). From skin permeability experiments disclosed herein, it clearly appears that ultrasound application successfully afforded introduction of higher amounts of Q-starch-HA complexes into deeper or lower layers of the skin, and a homogeneous distribution of these complexes therein.

The skin is the largest organ of the human body, it has a surface area of about 2 m² in healthy adults, a thickness of only a few millimeters and accounts for about 15% of adult's body weight. It is a heterogeneous multilayer tissue and contains almost one third of the circulating blood. The skin is a barrier to physical and chemical penetration from the environment to the body and has several functions including protection and resistance against environmental aggression, protection against infectious substances, protection from dehydration, and wound repair and rejuvenation. Two major tissue layers are conventionally recognized as constituting human skin. The outermost layer is the epidermis, and the second layer is the dermis.

The epidermis, about 0.07-1.4 mm thick, consists mainly of cells termed “keratinocytes” that are organized in five layers that represent the different stages of cell life in the epidermis. The sequence of layers from inside to outside is: (1) basal layer (stratum basale), composed of columnar cells arranged perpendicularly; (2) prickle-cell or spinous layer (stratum spinosum), composed of flattened polyhedral cells with short spines; (3) granular layer (stratum granulosum), composed of flattened granular cells; (4) clear layer (stratum lucidum), composed of several layers of clear, transparent cells in which the nuclei are indistinct or absent. In the epidermis of the general body surface, the clear layer is usually absent; and (5) horny layer (stratum corneum (SC)), composed of hexagonal, flattened, cornified, non-nucleated cells termed “corneocytes”, held together by lipids and desmosomes in what is commonly referred to as a brick-and-mortar structure. Desmosomes are specialized inter-corneocyte linkages formed by proteins, and together with the lipids, they maintain the integrity of the SC. The corneocytes in this outermost surface of the epidermis are dead, filled with keratin and form a tough and hydrophobic (13% of water) protective layer (also known as keratin layer). The lipids form several bilayers surrounding the corneocytes. The stratum corneum contains 15 to 20 layers of corneocytes, and, in its dry state, has a thickness of 10 to 15 μm. When hydrated, the stratum corneum considerably swells, and its thickness may reach up to 40 μm, accompanied with an increased permeability.

Stratum corneum is the main transport barrier for external substances. In addition, SC prevents water loss from the skin surface. It also participates in immunological and inflammatory processes. Considering its barrier characteristics and water resistance, the stratum corneum is the main layer that limits drug absorption through the skin.

The cells in the layers underneath the SC divide to replenish the supply. The epidermis does not have vascularization, thus living keratinocytes in the epidermis layers get nourishment from the dermis, which is separated from the epidermis by a basement membrane (the dermo-epidermal junction (DEJ)).

Dermis (the true skin) is the fibrous inner layer of the skin just beneath the epidermis, derived from the embryonic mesoderm, varying from 0.05 cm to 0.3 cm in thickness. The dermis comprises fibroblasts, histiocytes, and mast cells, its composition is mainly fibrous, consisting both of collagen and elastic fibers that are produced by the fibroblasts. Between the fibrous components lies an amorphous extracellular “ground substance” containing glycosaminoglycans such as hyaluronic acid, proteoglycans, and glycoproteins. The dermis provides structure and resilience, flexibility and strength to the skin and protects the body from mechanical harm. The dermis helps in maintaining the epidermis properties as well as in repairing and restoring skin after injury.

The dermis is composed of two zones: a superficial thin layer that interdigitates with the epidermis (the stratum papillare, or papillary dermis), and the deeper and coarser stratum reticulare (or reticular dermis). The papillary dermis is richly supplied with blood and lymphatic vessels and nerves and nerve endings. The reticular dermis is in contact with the subcutaneous (the innermost layer of the skin), it consists of thick collagen fibers that provide strength and elasticity to the skin, contains hair follicles, sweat glands, and sebaceous glands.

Collagen types I and II account for approximately 75% of the dry weight of the dermis.

The major route of skin permeation is through the intact epidermis, and two main pathways have been identified: the intercellular route through the lipids of the stratum corneum, and the transcellular route through the corneocytes. In both cases, a drug must diffuse into the intercellular lipid matrix, which is recognized as the major determinant of drug absorption by the skin. Drug transport in the skin can be seen as a process involving several steps: (a) dissolution and release of drug from the formulation; (b) drug partitioning into the stratum corneum; (c) drug diffusion across the stratum corneum, mainly through intercellular lipids; (d) drug partitioning from the stratum corneum into viable epidermis layers; (e) diffusion across the viable epidermis layers into the dermis; and (f) drug absorption by capillary vessels, which achieves systemic circulation. Being a barrier to water loss and permeation of substances from the environment, the SC provides mechanical protection to the skin that prevents efficient penetration of large molecules (>500 kDa). The main transdermal delivery routes of large molecules are thus often invasive, for example injections.

Glycosaminoglycans (GAGs), also known as mucopolysaccharides, are negatively charged polysaccharide compounds, containing amino sugars or monosaccharides in which the —OH group is replaced by an NH₂ group, such as D-glucosamine and D-galactosamine. They are composed of repeating disaccharide units and their functions within the body are widespread and determined by their molecular structure. For example, GAGs play a key role in cell signaling and in vast number of biochemical processes. Some of these processes include, for example, regulation of cell growth and proliferation, promotion of cell adhesion, anticoagulation, and wound repair. The four primary groups of GAGs are classified based on their core disaccharide units and include heparin/heparan sulfate, chondroitin sulfate/dermatan sulfate, keratan sulfate, and hyaluronic acid. Variations in the type of monosaccharides and presence or absence of modification by sulfation results in the different major categories of GAGs.

In one aspect, the present disclosure relates to a non-invasive method for transdermal delivery of one or more glycosaminoglycans (GAGs) in a subject in need thereof, the method comprising the steps of:

(a) applying ultrasound treatment to a skin surface of the subject for a period of from about 5 sec to about 5 min; and

(b) topically administrating to the ultrasound-treated skin surface one or more GAGs;

(c) optionally, applying a further ultrasound treatment to the skin surface for a period of from about 5 sec to about 5 min; and

(d) optionally, topically administrating to the ultrasound-treated skin surface, a further amount of one or more GAGs, thereby transdermally delivering the GAGs in a non-invasive manner.

Usually, but not necessarily, the GAGs transdermally delivered upon pre-application of ultrasound is of a molecular weight <500 kDa. Higher molecular weight GAGs, e.g., GAGs of molecular weights in the range of from 500 kDa to 8000 kDa, may be delivered, in accordance with the present disclosure, by assembling or complexing thereof with certain carrier polymers such as polysaccharides, optionally functionalized polysaccharides such as, but not limited to, starch, chitosan, pectin, cellulose, dextran, or galactan.

Such complexes have been previously utilized by the present inventors as non-viral carriers of microRNA (miRNA) for the treatment of psoriasis, small interfering RNA (siRNA) for the treatment of ovarian cancer, and PI3P for the treatment of hepatic insulin resistance (see, e.g., Amar-Lewis et al., Journal of Controlled Release 185:109-120, 2014; Lifshiz Zimon et al., Journal of Controlled Release 284:103-111, 2018). The use of polysaccharides as delivery vectors is considered advantageous due to their natural characteristics such as biodegradability, biocompatibility, low immunogenicity, and minimal cytotoxicity. Particularly, starch can be carefully designed and characterized in terms of molecular weight and modifications in order to address safety and efficiency issues in delivery.

In some embodiment, the carrier polysaccharide utilized in a contemplated method is a functionalized starch.

Starch is any of a group of polysaccharides of the general formula, (C₆H₁₀O₅)ₙ; it is the chief storage form of carbohydrates in plants and one of nature's energy reserves. Starch is a biodegradable polymer made up of D-glucose residues consisting of 20% amylose and 80% amylopectin. Amylose contains α-1,4 linkages, and amylopectin contains additional α-1,6 linkages. It is found chiefly in seeds, fruits, tubers, roots, and stem pith of plants, notably in corn, potatoes, wheat, and rice, and varying widely in appearance according to source but commonly prepared as a white amorphous tasteless powder. Starches possess many favorable characteristics such as low toxicity, biocompatibility, stability, low cost, hydrophilic nature and availability of reactive sites for chemical modifications.

In order for starch to be an effective carrier of GAGs, it needs to undergo certain modifications (also referred to herein as “functionalization”), for example, attaching positively charged groups thereto, since starch is an electrically neutral polysaccharide. The term “quaternized starch” (Q-starch) refers to a starch molecule having a backbone that has undergone certain modifications, such as substitution or addition, of at least one quaternary moiety or quaternary group. A quaternary moiety or group is defined herein as a cation consisting of a central positively charged atom with four substituents. Such a cation is also referred to herein as “quaternary cation”. A “quaternary compound”, as defined herein, is a compound that is or has a quaternary cation. The best-known quaternary compounds are quaternary ammonium salts, having a positively charged nitrogen atom at the center (N⁺R₄; R is a substituent). Other examples include substituted phosphonium salts (R₄P⁺), and substituted arsonium salts (R₄As⁺) like arsenobetaine. For example, quaternized potato starch may be obtained by substitution with a quaternary group, providing Q-starch with cationic properties. Q-starch may bind molecules bearing negatively charged groups by self-assembly formation of complexes.

In the context of embodiments described herein, Q-starch refers mostly to starch quaternized by substitution with one or more quaternary amine moieties.

The present disclosure, in a further aspect, relates to a non-invasive method for facilitating penetration of one or more GAGs having molecular weights of from 500 kDa to 8000 kDa transdermally to a deep layer of the skin, i.e., through the stratum corneum (SC) into deeper layers of the epidermis and downwards, e.g., to the dermis, the method comprising the steps of:

(a) applying ultrasound treatment to a skin surface of the subject for a period of from about 5 sec to about 5 min;

(b) topically administrating to the ultrasound-treated skin surface one or more GAGs complexed with at least one polysaccharide;

(c) optionally, applying a further ultrasound treatment to the skin surface for a period of from about 5 sec to about 5 min; and

(d) optionally, topically administrating to the ultrasound-treated skin surface, a further amount of GAG-polysaccharide complex(es), thereby facilitating penetration of the GAGs into deep layers of the skin.

Steps (c) and (d) may be repeated once, twice, three times or more, as needed.

“Deep layer of the skin”, as referred to herein, is a layer beneath the stratum corneum, for example, an inner epidermis layer such as stratum lucidum, stratum granulosum, stratum spinosum or the basal layer, and/or the dermis layer. “Deep layer of the skin” also refers to a depth of from 0 to about 2000 μm beneath the stratum corneum, for example from about 0 to about 10 μm, from about 5 μm to about 20 μm, from about 10 μm to about 30 μm, from about m to about 40 μm, from about 30 μm to about 60 μm, from about 50 μm to about 80 μm, from about 60 μm to about 100 μm, from about 80 μm to about 120 μm, from about 100 μm to about 150 am, from about 140 μm to about 200 am, from about 180 μm to about 250 am, from about 200 μm to about 270 am, from about 250 μm to about 300 am, from about 280 m to about 350 am, from about 320 μm to about 400 am, from about 250 μm to about 500 am, from about 450 μm to about 600 am, from about 500 μm to about 800 am, from about 650 μm to about 900 am, from about 800 μm to about 1000 am, from about 900 μm to about 1500 am, or from about 1000 μm to about 1800 μm beneath the SC, and any subranges and individual depths therebetween.

Glycosaminoglycans that may be transdermally delivered using a contemplated method described herein include, but are not limited to, heparin, heparan sulfate, chondroitin sulfate, dermatan sulfate, keratan sulfate, and hyaluronic acid. In some embodiments, any of these the GAGs has a molecular weight of from 500 kDa to 5000 kDa.

Heparan sulfate is known as a pharmacological target for cancer treatment. Noteworthy functions of heparan sulfate include extracellular matrix (ECM) organization and modulation of cellular growth factor signaling by acting as a bridge between receptors and ligands. In the extracellular matrix, heparan sulfate interacts with many compounds including collagen, laminin, and fibronectin to promote cell to cell and cell to extracellular matrix adhesion. In the setting of malignancy such as melanoma, degradation of heparan sulfate in the extracellular matrix by the action of the enzyme heparanase leads to migration of malignant cells and metastasis. This mechanism makes heparanase and heparan sulfate viable pharmacological targets for prevention of cancer metastasis.

Heparin is used as an anticoagulant by a mechanism that involves its interaction with the protein antithrombin III (ATIII), leading to a conformational change in ATIII that enhances its ability to function as a serine protease inhibitor of coagulation factors. Different molecular weights of heparin have been shown to exhibit various clinical anticoagulation intensities.

Chondroitin sulfate is known for its clinical use as a disease-modifying osteoarthritis drug (DMOAD), particularly for symptomatic pain relief and structure modification in osteoarthritis (OA). The pain-relieving properties of chondroitin sulfate in OA relate to its anti-inflammatory properties. One of the leading pathophysiological causes of OA relates to loss of chondroitin sulfate from articular cartilage in joints, leading to inflammation and catabolism of cartilage and subchondral bone. The structure-modifying role of chondroitin sulfate in OA is due to its role in stimulating type II collagen and proteoglycan (PG) production in both articular cartilage and the synovial membrane. This anabolic effect of chondroitin sulfate prevents further tissue damage and remodeling of synovial tissues.

Keratan sulfate has functional role in both the cornea and the nervous system. The cornea comprises the richest known source of keratan sulfate in the body, followed by brain tissue. The role of keratan sulfate in the cornea includes regulation of collagen fibril spacing that is essential for optical clarity, as well as optimization of corneal hydration during development based on its interaction with water molecules. As with other GAGs, the degree of sulfation of keratan sulfate determines its functional status. Keratan sulfate has also been shown to play an important regulatory role in the development of neural tissue. Various subgroups of keratan sulfate in the brain have key roles for stimulating the growth of microglial cells and the promotion of axonal repair following injury.

Hyaluronic acid (HA) or hyaluronan has the simplest structure of all GAGs, being a long, homogeneous, unbranched polysaccharide consisting of two repeating disaccharide units: D-glucuronic acid and N-acetyl-D-glucosamine linked together through alternating β-1,4 and β-1,3 glycosidic bonds. The number of repeating disaccharide units in a HA molecule can reach 10,000 units or more in the human body. Hyaluronic acid is the main component of extracellular matrix, being mostly abundant in the skin (approximately 50% of the total HA resides in the skin, both in the dermis and the epidermis) and accounting for 15% of the total body mass. Hyaluronic acid is present in all tissues and fluids of the body including vitreous of the eye, joints, cornea the umbilical cord, and synovial fluid. Hyaluronic acid plays a vital role in the synthesis of extracellular matrix molecules and epidermal cell interaction with the surrounding environment. It modulates cellular immunity by preventing infections and impeding allergic phenomena.

Hyaluronic acid production is controlled by fibroblasts, keratinocytes, or chondrocytes. In tissues such as skin and cartilage, where HA comprises a large portion of the tissue mass, HA synthesis level is very high. Hyaluronan has a dynamic turnover rate with a half-life of 3 to 5 min in the blood, less than a day in the skin, and 1 to 3 weeks in the cartilage. It degrades into fragments of varying sizes by hydrolysis effected enzymatically by hyaluronidases, or non-enzymatically by a free-radical mechanism in the presence of reducing agents such as ascorbic acid, thiols, ferrous, or cuprous ions, a process that requires the presence of molecular oxygen.

Hyaluronic acid is best-known for its capability of attracting water molecules. In physiological solution, the carboxyl groups of HA are negatively charged (anionic), and HA can form salts with mobile cations. These salts are highly hydrophilic and, consequently, surrounded by water molecules. The highly polar structure of HA makes it capable of binding 10000 times its own weight in water. Water molecules link to HA carboxyl and acetamido groups via H-bonds that stabilize the secondary structure of the biopolymer, described as a single-strand left-handed helix with two disaccharide residues per turn (two-fold helix). In aqueous solution, these two-fold helices form duplexes, i.e., a p-sheet tertiary structure, due to hydrophobic interactions and inter-molecular H-bonds, which enable the aggregation of polymeric chains with the formation of an extended meshwork. As molecular weight (MW) and concentration increase, the HA networks are strengthened. Due to these characteristics, HA plays a key role in lubrication of synovial joints and wound healing processes.

Hyaluronic acid has a rather wide molecular size/weight range (10⁵-10⁷ Da), occurring in a vast number of configurations and shapes, depending on its size, salt concentration, pH and associated cations. The biological functions of HA are strongly dependent upon its size: high-molecular-weight hyaluronic acid (HMW HA) chains (>5×10⁵ Da) are space-filling, antiangiogenic, immunosuppressive, inhibit cell proliferation and migration of vascular endothelial cell, and are usually applied in the pharmaceutical field in various applications (e.g., cancer treatment, osteoarthritis treatment, ophthalmic surgery, plastic surgery, drug delivery, and wound healing); medium size HA chains of lower than 500 kDa (e.g., between 2×10⁴ to 10⁵ Da) are involved in ovulation, embryogenesis, and wound repair; low molecular weight HA (LMW HA) chains of less than 100 kDa (e.g., between 6×10³ to 2×10⁴ Da) are inflammatory, immunostimulatory, and angiogenic, while small HA oligomers (from 400 to 4000 Da) are anti-apoptotic and inducers of heat shock proteins. Low molecular weight HA and smaller oligosaccharides may be produced by naturally in the body or artificially by controlled depolymerization of HMW HA using physical treatment (thermal treatment, pressure), irradiation, ultrasound application, acid treatment, radical oxidation, and enzymatic hydrolysis with hyaluronidase.

Hyaluronic acid is generally applied in the cosmetics and food industries and used exogenously by clinicians for promotion of tissue regeneration and skin repair. It has demonstrated safety and efficacy for these purposes and has been approved by the Food and Drug Administration (FDA) as a dermal filler. Some HA-based products are already on the market and/or have already a consolidated clinical practice, while others are currently under research to confirm their effectiveness.

In cosmetic products, HA shows promising efficacy in promoting skin tightness, elasticity, and improving aesthetic scores. For example, utilization of HA in cosmetic formulations as a moisturizing active ingredient that restores the physiological microenvironment typical of youthful skin, is well-known and widely used. For cosmetic utilization, HA is classified by its molecular weight. Hyaluronic acid of 20-300 kDa is able to penetrate the stratum corneum, and HA of 5 kDa even permeates deeper into the epidermis, whereas higher molecular weights of HA (500-1500 kDa) stay normally on the surface of the skin and are not able to penetrate the stratum corneum (SC).

Hyaluronic acid utilized in embodiments described herein encompasses any form of HA commercially available or custom-made, produced by any of the techniques known in the art such as, but not limited to, extraction from animal sources or microorganism fermentation (e.g., fermentation of strains of bacteria Streptococci).

Some embodiments pertain to the use of chemically modified HA. Chemical modifications of HA mainly involve two functional sites: the hydroxyl (probably the primary alcoholic function of the N-acetyl D glucosamine) and the carboxyl groups. These functional groups can be modified through two techniques, which are based on the same chemical reactions, but lead to different products: conjugation and crosslinking. Conjugation consists of grafting one or more monofunctional molecule onto the HA chain, each forming a single covalent bond, while crosslinking employs polyfunctional compounds that link together different chains of native or conjugated HA by two or more covalent bonds. Crosslinked hyaluronan can be prepared from native HA (direct crosslinking) and/or from HA conjugates (i.e., HA covalently linked to one or more functional groups). In direct crosslinking of native HA molecules, hydroxyl and carboxyl groups can be crosslinked via ether linkages and ester linkages, respectively. In some embodiments, HA is chemically modified before crosslinking thereof so as to introduce other chemically reactive groups. For example, HA may be treated with acid or base such that it will undergo at least partial deacetalisation, resulting in the presence of free amino groups which can then be crosslinked via an amide (—C(O)—NH—); imino (—N═CH—) or secondary amine (—NH—CH—) bond. An imino linkage can be converted into an amine linkage in the presence of a reducing agent.

Conjugation and crosslinking are generally performed for different purposes. For example, conjugation provides crosslinking with a variety of molecules to obtain, e.g., carrier systems with improved drug delivery properties or pro-drugs. Crosslinking further improves the mechanical, rheological and swelling properties of HA and reduces its degradation rate and may provide HA derivatives with a longer residence time at the site of application and greater release properties. A higher degree of cross-linking reduces the water absorption capacity of the cross-linked HA, resulting in greater stability in aqueous solution. In addition, double cross-linked HA exhibits greater stability against degradation by hyaluronidase, and against degradation due to free radicals, thus affording an increased biostability. For example, crosslinked HA has been used for cosmetic application in the field of skin aging.

The term “hyaluronic acid”, as used herein, encompasses native HA in any molecular weight known in the art as well as HA derivatives which include any chemically of physically modified HA such as, but not limited to, HA conjugates and crosslinked HA.

Hyaluronic acid homeostasis changes with aging as well as due to external and internal processes and agents such as exposure to sun which cause degradation of HMW HA. The HA content of the dermis is significantly higher than that of the epidermis. Both epidermal and dermal cells can synthesize HA throughout our lifetime. However, the skin cells lose their ability to produce optimal amounts of HA during aging processes. The major histochemical change observed in senescent skin is the marked decrease in epidermal HA, while HA is still present in the dermis. Thus, the epidermis loses the principal molecule responsible for binding and retaining water molecules, resulting in loss of skin moisture. In the dermis, the major age-related change is the increasing avidity (functional affinity) of HA with tissue structures with the concomitant loss of HA extractability. This parallels the progressive cross-linking of collagen and the steady loss of collagen extractability with age. The decline in HA production is also accompanied by a decreased suppleness, reduced elasticity, and loss of skin tone that characterizes aged skin.

In order to retain the aesthetic appearance of the skin, and treat the signs of “dermatological” aging, it is recommended to keep “refilling” skin with HA from adolescent age onwards. The treatments available today for adding HA to the skin are serums, injections and oral intake. It is already known that HA taken orally does not show any benefit to skin appearance, because skin cells are not able to extract HA from the bloodstream. Topical application of HMW native HA (>6×10⁵ Da) is challenging because it does not efficiently penetrate the deeper skin layers, mainly due to its large size. Instead, it forms films that act as barriers against moisture loss. In addition, due to its good gelling properties, topical application of HA leads to a hydration effect in the uppermost layer of the SC, and the water that accumulates may cause the compact structure of the horny layer to swell and open, leading to an increase in the extent of HA penetration to upper layers of the epidermis. In this way, HMW HA has positive effect on hydration of upper epidermis layers, which is manifested by lower trans-epidermal water loss. Skin hydration capacity is dependent on HA molecular size, and for this reason, HMW HA (about 1 MDa) is usually added to cosmetic formulations.

Yet, however, penetration of HA, particularly HMW HH, into the deeper layers of the skin is very slow. Penetration properties (and thus anti-aging effect) of HMW HA applied topically can be improved by combination of HA with skin penetration carriers. For most cases, and for all HA application into deeper skin layer, HA and particularly crosslinked HA, is injected, in a rather painful application procedure that may sometimes cause inflammatory complications and bacterial infections.

In a further aspect, the present disclosure relates to a non-invasive method for facilitating transdermal delivery of hyaluronic acid by applying ultrasound (US) treatment to the skin prior to topically applying HA. A contemplated method comprises at least the following steps:

(a) applying ultrasound treatment to a skin surface of the subject for a period of from about 5 sec to about 5 min; and

(b) topically administrating to the ultrasound-treated skin surface a solution of hyaluronic acid;

(c) optionally, applying a further ultrasound treatment to the skin surface for a period of from about 5 sec to about 5 min; and

(d) optionally, topically administrating to the ultrasound-treated skin surface, a further amount of hyaluronic acid solution, thereby facilitating transdermal delivery of hyaluronic acid in the subject.

In some embodiments, a contemplated method facilitates transdermal delivery of HA of molecular weight in the range of 300-800 kDa to the epidermis, primarily to upper epidermis layers. Penetration of HA of higher MW may also benefit from a prior application of US to the treated skin site.

It has been discovered by the present inventors that in order to increase skin permeation to HA of even higher MW and penetration thereof to even deeper layers of the epidermis, ultrasound application may be combined with HA complexation, e.g., assembling HA with a carrier such as a polysaccharide, as described herein for GAGs transdermal delivery.

Embodiments of the present disclosure relate to a non-invasive method for enabling or advancing penetration of high MW hyaluronic acid into deep layers of the skin of a subject in need thereof, the method comprising the steps of:

(a) applying ultrasound treatment to a skin surface of the subject for a period of from about 5 sec to about 5 min;

(b) topically administrating to the ultrasound-treated skin surface, a complex comprising hyaluronic acid and a polysaccharide, thereby advancing penetration of hyaluronic acid into deep layers of the skin in the subject.

The method is useful for transdermal delivery (i.e., delivery by the skin and/or intradermal delivery) of HA of molecular weight >500 kDa, for example, a molecular weight in the range of from 500 kDa to 8000 kDa, from 1000 kDa to 5000 kDa, or from 500 kDa to 3000 kDa.

Optionally, steps (a) and (b) of a disclosed method may be repeated at least one more time (e.g., once, twice, three times or more) as needed in order to achieve efficient penetration of hyaluronic acid into deep layers of the skin/Namely, after the first US application, a further ultrasound treatment may be applied to the treated skin surface for a period of from about 5 sec to about 5 min, optionally followed by topically administrating a further amount of the hyaluronic acid-polysaccharide complex. In some embodiments, a second or third US application to the treated skin is not concomitantly followed by additional HA administration.

In some embodiments, a contemplated non-invasive method for facilitating transdermal delivery of high or low molecular weight hyaluronic acid into deep layers of the skin is utilized for treating or preventing skin aging processes in a subject in need thereof.

“Preventing or treating skin aging processes”, as used herein, is at least one of maintaining skin hydration, restoration, or improvement of collagen production, delaying aging process such as wrinkling, or reducing aging indicators associated with loss of mechanical properties such as loss of elasticity and skin atrophy.

Skin atrophy is a common manifestation of aging and is frequently accompanied by ulceration and delayed wound healing. Atrophic skin displays a decreased HA content and expression of the major cell-surface hyaluronate receptor, CD44. With an increasingly aging patient population, management of skin atrophy is becoming a major challenge in the clinic, particularly in light of the fact that there are no effective therapeutic options at present.

In some embodiments, hyaluronic acid is complexed with a quaternized starch (Q-starch) carrier as defined herein. In some embodiments, the Q-starch is a low molecular weight potato starch (26.7 kDa) modified with a quaternary amine that generates self-assembled nano-sized complexes with HA (herein also termed “nano-complexes”).

Ultrasound (for brevity denoted herein as “US”) is a sound wave with frequency above 18 KHz, which is the limit of human hearing. The ultrasound wave is a longitudinal wave, i.e., the direction of propagation is the same as the direction of oscillation. Ultrasound wave is also termed “pressure wave” since it causes compression and expansion of the medium, leading to pressure variations in the medium. The ultrasound wave frequency (f) is the number of pressure variation cycles in the medium per unit time (vibrating rate) measured in Hertz (Hz), wherein each cycle is composed of compression and rarefaction. The wave amplitude (A) describes the maximum local pressure measured in Pascal units (Pa).

A typical ultrasound induction apparatus contains a piezoelectric transducer which converts electrical signals into ultrasound waves. By applying an alternating voltage across a piezoelectric material, the material oscillates at the same frequency as the driving current. The transducer can operate in continues mode (repeated cycles) or in pulse mode (cycles separated in time with gaps with no signal).

Ultrasound treatments are usually noninvasive and focused. They can be modified by changing various parameters such as the US frequency, intensity, amplitude, acoustic pressure, pulses duration and period. Thus, although US waves propagate through multiple tissue layers, they can be focused or targeted to a small volume in a specific organ or tissue inside the body for treatment purposes, and the transferred energy, which in some conditions can lead to tissue heating and destruction, can be concentrated or localized at a predetermined specific target or spot in the tissue or organ with no adverse effects to the entire tissue or adjacent organs.

The effects of US on biological tissues include mainly thermal heating, acoustic cavitation, and acoustic streaming. Thermal heating is the result of US waves transiting through the medium. The US sound waves are absorbed by the medium and induce the formation of heat which can be conducted, convected or radiated. Thermal effects increase with frequency and are most significant at megahertz frequencies. Low-frequency ultrasound has shown the ability to significantly increase skin permeability and enable the delivery of various substances through the skin. The main mechanism that accounts for ultrasound's ability to increase skin permeability is acoustic cavitation, which can momentarily induce growth and oscillations of present air pockets in the corneocytes of the stratum corneum.

The term “cavitation”, as used herein, refers to a phenomenon in which rapid changes of pressure in a liquid lead to the formation of small vapor-filled cavities in places where the pressure is relatively low. Expansion cycles in a medium exert a negative pressure and pull the molecules apart. When pressure amplitude exceeds the tensile strength of liquid in the rarefaction regions, small vapor-filled cavities are formed. When subjected to higher pressure, these cavities, also termed “cavitation bubbles” or “voids”, collapse and can generate an intense shock wave. Cavitation in a liquid medium, namely, the formation of gaseous cavities, may be the consequence of US-induced pressure variations in the medium. The cavitation threshold intensity depends on the medium (temperature, pressure, and dissolved gas concentration) and the sound wave's physical parameters.

The term “acoustic cavitation”, as used herein, refers to the formation of gas bubbles, and activity (growth, oscillations, or collapse) of existing gas bubbles in a medium exposed to ultrasound waves. When existing bubbles in a liquid medium are exposed to ultrasound, they oscillate or collapse and generate acoustic emission, i.e., cavitating gas bubbles are secondary sources of acoustic sound. There are two types of acoustic cavitation: stable and inertial (also termed “transient cavitation”). Stable cavitation is prolonged oscillations (for a considerable number of cycles) of gas bubbles in response to pressure changes. The bubbles expand during the rarefaction phase and contract during the compression phase, oscillating about the equilibrium radius for several cycles. The stable oscillations create a liquid flow around the bubble, known as microstreaming, that induces shear stress. If the bubbles are located near a biological tissue such as skin, these shear stresses can cause pore formation and affect tissue permeability.

Inertial cavitation occurs in higher pressure amplitudes when the pressure amplitude is sufficiently high and reaches a critical value (the inertial cavitation threshold). The bubbles grow and collapse violently; during the collapse, symmetrical shockwaves with high pressure (exceeding 10 Kbar) and temperature can be generated in the close area of the collapsing bubble. When the bubbles are close to a solid surface, the collapse is asymmetrical, and a liquid jet may be created. When the collapse occurs near a biological tissue, it can cause membrane perforation, reversible pore formation and/or blood vessel permeabilization.

The acoustic cavitation phenomenon is utilized in embodiments described herein for increasing skin permeability, affording transdermal delivery of various GAGs via the skin (for systemic delivery) and between skin layers (intradermal delivery). Without wishing to be limited by theory, it is assumed that cavitation creates disorder in the stratum corneum lipids, and in the region of disordered lipids, water penetrates and promotes the formation of aqua channels. These channels in the intercellular lipids of the SC enable the transport of large molecules. Three modes of cavitation effect induce by US are assumed: shockwaves, impact of microjets on the SC, and microjet penetration into the SC. Both microjets and shockwaves might be responsible for the SC permeability enhancement effect, with microjets being significantly more effective in increasing permeability of the skin (see, e.g., Tezel and Mitragotri, Biophys J., 2003; 85(6):3502-3512; Azagury et al, Adv Drug Deliv Rev., 2014; 72:127-143; Wolloch and Kost, J Controlled Release., 2010; 148(2):204-211).

In some embodiments, US is being applies in combination with simultaneous topical application of one or more skin penetration enhancers such as, but not limited to, surfactants ranging from hydrophobic agents such as oleic acid to hydrophilic sodium lauryl sulfate (SLS). Surfactants are found in many existing therapeutic, cosmetic, and agro-chemical preparations, and in recent years have been employed to enhance the permeation rates of several drugs via transdermal route. Surfactants have effects on the permeability characteristics of several biological membranes including skin. They have the potential to solubilize lipids within the stratum corneum. The penetration of the surfactant molecule into the lipid lamellae of the stratum corneum is strongly dependent on the partitioning behavior and solubility of surfactant.

It has been found by the present inventors that simultaneous application of US (e.g., 3 W/cm², 0.5 s ON and 0.5 s OFF) and SLS (1% solution) led to changes in the pH of the SC, which affected both the structure of the lipid layers and the solubility of SLS inside the skin.

Such simultaneous application may result in a synergistic effect of US and SLS on SC permeability.

In some embodiments, US is being applied in combination with SLS so as to increase the skin permeability to GAGs in general and to HA in particular.

Synthetic microbubbles particularly designed and fabricated as cavitation nuclei (cavitation source) may be used in a contemplated method. Combining the synthetic microbubbles with ultrasound waves may be utilized for opening of various biological barriers, including enhancing transdermal delivery of GAGs.

### Complexes of Hyaluronic Acid and Modified Starch

An aspect of the present disclosure relates to a complex of hyaluronic acid and a chemically modified starch. Such a complex serves as a carrier to facilitate delivery of hyaluronic acid into deep layers of the skin as defined herein. A disclosed complex is particularly useful for delivering high molecular weight hyaluronic acid.

There are three types of functional groups in hyaluronic acid that can be used for coupling thereof with a carrier polymer: anomeric carbonyl group, the hydroxyl groups or the carboxyl groups. Depending on the targeted group of hyaluronic acid and the functional groups in the carrier, the conjugates can be obtained by a direct reaction between both macromolecules. In most cases, a modification of hyaluronic acid and/or of the carrier polymer is required as a preliminary step to incorporate new functional reactive groups in order to facilitated conjugation and/or formation of stable complexes. The synthetic strategy for coupling a modified polymer to hyaluronic acid is usually selected depending on the functional groups displayed by the former. Since hyaluronic acid is negatively charged at physiological pH, its complexation with positively charged polymers such as polyaniline, chitosan, poly(β-amino ester), poly D-lysine is known and has been used for the synthesis of HA-based nanocarriers. Biodegradable polymers are usually the preferred option.

A complex, as referred to herein, encompasses the product of conjugation, self-assembling, crosslinking and the like, between HA and carrier polymer, as well as encapsulation of HA by a carrier polymer. Complexes contemplated herein are usually of nano-size dimensions and are also referred to herein as nano-complexes or nano-carriers.

Embodiments described herein pertain to complexes of hyaluronic acid with quaternized starch (Q-starch) obtained by self-assembly of the two polymers. Modified starch is obtained by covalently attaching (i.e., substituting) at least one quaternary amine group thereto. Q-starch may be obtained by reaction with 2,3 epoxypropyltrimethyl ammonium chloride or 3-chloro-2-hydroxypropyltrimethyl ammonium chloride (CHMAC).

In some exemplary embodiments, the quaternary amine moiety is (CH₃)₃—N⁺—.

Complexes of and Q-starch and hyaluronic acid (herein designated “Q-starch-HA”) are designed and fabricated so as to feature a desired molar ratio between the positively charged chemical moieties of the modified starch and the negatively charged carboxyl groups of hyaluronic acid (herein designated “N/O molar ratio”, “N/O ratio”, or simply “N/O”). The N/O molar ratio affects complexation and/or transdermal penetration efficacy. Contemplated Q-starch-HA may feature N/O ratios in a range of from about 0.20 to about 3.50, depending, inter alia, on the molecular weight of hyaluronic acid and/or the type and MW of the Q-starch. For example, N/O may be any ratio in a range of from about 0.20 to about 0.40, from about 0.22 to about 0.26, from about 0.25 to about 0.35, from about 0.30 to about 0.45, from about 0.40 to about 0.60, from about 0.50 to about 0.70, from about 0.65 to about 0.80, from about 0.75 to about 0.90, from about 0.80 to about 1.00, from about 0.85 to about 1.10, from about 1.00 to about 1.20, from about 1.10 to about 1.40, from about 1.25 to about 1.50, from about 1.35 to about 1.65, from about 1.50 to about 1.85, from about 1.70 to about 2.00, from about 2.10 to about 2.50, from about 2.30 to about 2.65, or from about 2.60 to about 3.00, and any subranges and individual values therebetween.

In some embodiments, N/O is in the range of from about 0.22 to about 0.50, form about 0.25 to about 1.50 or from about 1.00 to about 2.50. In some embodiments, N/O is 0.25.

### Pharmaceutical Compositions

In a further aspect, the present disclosure relates to compositions comprising one or more Q-starch-HA complexes as described herein and physiologically acceptable excipients. The disclosed composition may be a cosmetic composition and have cosmetic utilization and/or a pharmaceutical or therapeutic composition and have a therapeutic utilization. In some embodiments, the composition is formulated for transdermal administration and comprises a physiologically acceptable carrier.

The terms “pharmaceutical composition” and “cosmetic composition” as used herein, refer to a composition essentially comprising at least one Q-starch-HA complex, which may be adapted for clinical or cosmetic utilization, respectively such as, but not limited to, therapeutic or anti-aging utilization. “Formulation”, as used herein, refers to any mixture of different components or ingredients, at least one of which is a Q-starch-HA complex, prepared in a certain way, i.e., according to a particular formula so as to be applicable for administration to a subject. Such formulation is termed herein “Q-starch-HA formulation”. For example, a Q-starch-HA formulation may be formulated for topical or transdermal administration and may include one or more Q-starch-HA complexes combined or formulated together with, for example, one or more carriers, excipients, penetration enhancers, stabilizers and the like.

As used herein, the terms “pharmaceutically acceptable”, “pharmacologically acceptable” and “physiologically acceptable” are interchangeable and mean approved by a regulatory agency of the Federal or a state government or listed in the U.S. Pharmacopeia or other generally recognized pharmacopeia for use in animals, and more particularly in humans. These terms include formulations, molecular entities, excipients, carriers and compositions that do not produce an adverse, allergic or other untoward reaction when administered to an animal, or a human, as appropriate. For human administration, preparations should meet sterility, pyrogenicity, general safety and purity standards as required by, e.g., the U.S. Food and Drug Administration (FDA) agency, and the European Medicines Agency (EMA).

Herein the term “excipient” refers to an inert substance added to a pharmaceutical composition or formulation to further facilitate process and administration of the active ingredients. “Pharmaceutically acceptable excipients”, as used herein, encompass approved preservatives, antioxidants, surfactants (e.g., Tween®-20, Tween®-40, Tween®-60 and Tween®-80), buffers, coatings, isotonic agents, absorption delaying agents, penetratin enhancers, carriers and the like, that are compatible with pharmaceutical administration, do not cause significant irritation to an organism and do not abrogate the biological activity and properties of a possible active agent. Physiologically suitable carriers in liquid formulations may be, for example, solvents or dispersion media.

### Kits

In still a further aspect, the present disclosure relates to a kit comprising: (a) at least one Q-starch-HA complex or a Q-starch-HA formulation as defined herein; (b) means of applying ultrasound; and (c) optionally, instructions and means for administration of the complexed hyaluronic acid and/or the formulation to a subject in need thereof.

A contemplated kit is useful for enhancing non-invasive transdermal delivery of hyaluronic acid, e.g., high molecular (>1000 kDa) hyaluronic acid, particularly to deep layer of the epidermis and/or the dermis, which may find use in anti-aging treatment as well as any other treatment modality that utilizes hyaluronic acid.

It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

As used herein the term “about” refers to +10%.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this description, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the present disclosure in a non-limiting fashion. Generally, the nomenclature used herein, and the laboratory procedures utilized in the present disclosure include molecular, chemical, biochemical and/or microbiological techniques. Such techniques are thoroughly explained in the literature. Other general references are provided throughout this document. The procedures therein are believed to be well known in the art and are provided for the convenience of the reader.

### Materials

The following materials were purchased from Sigma-Aldrich Inc.: N-hydroxysulfosuccinimide sodium salt (Sulfo-NHS) (P56485); N-(3-dimethylaminopropyl)-N-ethyl carbodiimide hydrochloride (EDAC) (E1769); sodium chloride (NaCl) (S-0399); 20(N-morpholino)ethanesulfonic acid hydrate, (MES hydrate) (M2933); sodium hydroxide (S-0399); 3-chloro-hydroxypropyltrimethylammonium chloride (348287); phosphate buffered saline (PBS) (P4417); and sodium lauryl sulfate (SLS) (L5750). Acetone and ethanol were purchased from Bio-Lab. Sodium Hyaluronate (1500 KDa; 025693), was purchased from Life core Biomedical. Soluble starch (101252) was purchased from Merck. Prolong gold antifade reagent with DAPI (P36935) was purchased from Invitrogen. Full-thickness skin from porcine ears was purchased from the Institute of Animal Research, Lahav, Israel. Hylite™ Fluor 647 nm amine (81257) was purchased from Anaspec.

### (i) Hyaluronic Acid Labeling

The labeling of HA was performed as previously described in Sapir et al. (Biomaterials, 2011, 32.7: 1838-1847). The fluorescent labeling dye was covalently attached to HA via carbodiimide chemistry, creating an amide bond between the terminal amine groups on the fluorescent molecule and the carboxylic groups on the HA. Briefly, 10 ml of 0.2% (w/v) HA aqueous solution was prepared, and 426 mg 4-morpholineethanesulfonic acid monohydrate (MES-H₂O) and 200 mg NaCl were added to obtain a pH 6.5 solution. The mixture was stirred at room temperature for 10 min. The carboxylic groups on HA were activated by the addition of 38.4 mg/0.5 ml 1-ethyl (dimethylaminopropyl)-carbodiimide (EDAC) in double distilled water (DDW), and 21.6 mg/0.5 ml of the co-reactant N-hydroxysulfosuccinimide (sulfo-NHS) in DDW was added to stabilize the reactive intermediate. The mixture was stirred at room temperature for 3 h, and 1 mg/0.5 ml of Hylite™ Fluor 647 amine dye in DDW was added and stirring continued for an additional 12 h to ensure formation of an amide bond between the amine groups of the dye and the HA carboxylic groups. The synthesis product (labeled HA) was purified by a dialysis bag at molecular weight cutoff (MWCO) of 11 KDa that was placed in a vessel containing 5 L of distilled water (DW). The water was replaced 6 times with fresh DW during 3 days of dialysis. The dialyzed product was then dried by lyophilization for 72 h and stored desiccated at 4° C.

### (ii) Starch Quaternization

Modification or derivatization of starch by way of converting it into a cationic polymer is essential in order to enable self-assembly thereof with hyaluronic acid via electrostatic interactions between positively charged groups of the modified starch and negatively charged carboxylic groups of HA. Quaternary starch (Q-starch), namely starch modified by substitution with quaternary ammonium groups, is known as a biocompatible and biodegradable carrier molecule for gene delivery.

Starch modification with quaternary amine groups to obtain Q-starch was performed as previously described by Amar-Lewis et al. (Journal of controlled release 185:109-120, 2014), based on Geresh et al. (Carbohydr Polym. 43(1):75-80, 2000), and as schematically presented in Scheme 1 below. First, 500 mg of soluble potato starch (hydrolyzed potato starch, MW 26,765 Da) were dissolved in 10 ml of sodium hydroxide solution (0.19 g/ml) to obtain 50 mg/ml starch concentration. The solution was then stirred continuously for 30 min at room temperature. Nine grams (9 g, 0.029 mol, 7.8 ml) of the quaternization reagent 3-chloro-2-hydroxypropyltrimethyl-ammonium chloride (CHMAC) were dissolved in 20 ml of DW (0.32 g/ml) and added to the starch solution. The reaction mixture was continuously stirred for 24 h at room temperature. For product precipitation, one volume of the product was precipitated by adding 4 volumes of an acidified (1% HCl) mixture of ethanol and acetone (1:3 vol %). The precipitate was washed 4 times with ethanol, dissolved in a small volume (1-2 ml) of DW, and poured into an 11 kDa cutoff dialysis bag that was placed in a vessel containing 5 L of DW. Dialysis was performed in order to remove unreacted cationic reagent. The water was replaced 4 times with fresh DW during 48 h of dialysis. The dialyzed product was then freeze dried by lyophilization for 72 hours.

Starch quaternization reaction using the quaternary agent CHMAC is presented in Scheme 1:

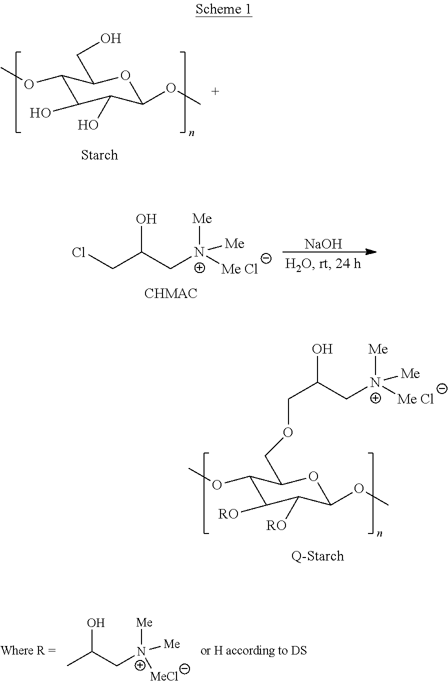

Quaternization of starch was confirmed by Fourier transform infrared spectroscopy (FT-IR) and Elementary analysis (EA). The measurements were obtained in a Thermo Nicolet™ FT-JR spectrophotometer (Nicolet™ iS™ 10 FT-JR spectrophotometer). Obtention of Q-starch was verified by the strong and new absorption bands that appeared at 3027 cm⁻¹ and 1478 cm⁻¹. These bands are related to the C—H and C—N stretching vibrations, respectively, of quaternary ammonium group (CH₃)₃N—. The rest of the bands were similar when compared with FT-TR spectrum of the unmodified starch. Samples were prepared in the form of potassium bromide (KBr) pellets.

Nitrogen content of Q-starch is a necessary parameter, since calculation of the ratio between positively charged amine groups on Q-starch (N) and negatively charged carboxylic groups on HA backbone (O) (N/O) is based on the amount of positive amine groups per chain of starch. Nitrogen atom weight percentage of Q-starch (N %) was evaluated by the EA method (see, for example, Jeffery et al., Vogel's textbook of quantitative, Chem. Anal., 302-303, 1989) and found to be 3.44%. According to calculations based on the quaternization of the 6′ position in each glucose monomer of starch, 4.2% is considered the maximum substitution. Nitrogen weight % in Q-Starch is calculated using Equation 1:

$$\frac{Mw of N ( 14 \frac{g}{mol} )}{Mw of ( 1 - starch ) ( 131.6 \frac{g}{mol} )} ⋆ 100 \% = N \% ( wt . )$$

The most significant advantage of using a quaternary amine as the substituted molecule is the polymer's electric charge which is almost independent of the solution's pH.

The quaternary starch retains its positive charge in a wide pH range, unlike chitosan complexes, which are highly dependent on pH and remain stable mainly under acidic conditions.

(iii) Q-Starch Labeling

Q-starch was labeled using 5-(4,6-dichlorotriazinyl) aminofluorescein (5-DTAF). First, 100 mg of Q-starch was dissolved in 3 ml of DDW, and the pH was adjusted with 1 M NaOH to pH 11-12, under stirring for 30 min at room temperature. After 30 minutes, 7.5 mg of 5-DTAF (dissolved in 0.3 ml dimethyl sulfoxide (DMSO)) were added to the Q-starch solution and stirred at room temperature for 24 h in the dark. The reaction mixture was then neutralized with 0.2 M HCl and poured into an 11 KDa cut-off dialysis bag. The labeled polysaccharide, Q-starch-5-DTAF, was separated from free 5-DTAF by extensive dialysis against PBS buffer solution (pH 7.5) for 72 hours, and then against DDW for 48 hours. The dialyzed product was then freeze-dried and lyophilized for 72 h to obtain the purified Q-starch-5-DTAF.

### (iv) Physical Characterization of HA, Q-Starch and Q-Starch-HA Complexes

Physical characterizations of the Q-starch-HA complexes at different N/O ratios were obtained using Zeta potential for measuring the surface charge, NanoSight for measuring diameter size, Dynamic Light Scattering (DLS) for measuring the hydrodynamic size, and cryogenic Transmitting Electron Microscopy (cryo-TEM) for measuring the size and geometry of the complexes.

(a) Zeta Potential (ζ-Potential)

Zeta potential (ζ-potential) is the charge that develops at the interface between a solid surface and its liquid medium. This potential, which is measured in MilliVolts, may arise by any of several mechanisms. Among these are the dissociation of ionogenic groups in the particle surface and the differential adsorption of solution ions into the surface region. The net charge at the particle surface affects the ion distribution in the nearby region, increasing the concentration of counterions close to the surface. Thus, an electrical double layer is formed in the region of the particle-liquid interface, consisting of two parts: an inner region that includes ions bound relatively tightly to the surface, and an outer region where a balance of electrostatic forces and random thermal motion determines the ion distribution. The potential in this region, therefore, decays with increasing distance from the surface until, at sufficient distance, it reaches the bulk solution value, conventionally taken to be zero.

In an electric field, each particle and its most closely associated ions move through the solution as a unit, and the potential at the surface of shear between this unit and the surrounding medium is known as the zeta potential. In other words, zeta potential is defined as the average electrostatic potential existing at the hydrodynamic plane of shear. When a layer of macromolecules is adsorbed on the particle's surface, it shifts the shear plane further from the surface and alters the zeta potential.

Measurement of ζ-potential is currently the simplest and most straightforward way to characterize the surface of charged colloids and is most relevant to the practical study and control of colloidal stability and flocculation processes.

Surface charge of hyaluronic acid, Q-starch and Q-starch-HA complexes were determined by zeta potential measurements using Zetasizer (ZN-NanoSizer, Malvern, England). Complexes were prepared as described in Example 1 below at different N/O ratios (e.g., 0.25-3), and diluted to obtain a final HA concentration of 26 mM in a volume of 1 ml DDW. Samples were transferred to U-tube cuvette (DTS1070, Malvern) and measured in automatic mode at 25° C. Smoluchowski model (Smoluchowski, Z. Phys. Chem., 1917, 92:129-168) was used for calculating the zeta potential. For each sample, the zeta potential value was presented as the average value of three runs (triplicates±standard deviation).

(b) Dynamic Light Scattering (DLS)

Dynamic light scattering (DLS) measures the temporal fluctuations of the light scattered due to the Brownian motion of particles, when a solution containing the particles is placed in the path of a monochromatic beam of light. Brownian motion of particles correlates with their hydrodynamic diameter. The smaller the particle, the faster it will diffuse. DLS is also known as photon correlation spectroscopy or quasi-elastic light scattering. This technique analyzes modulation of the intensity of scattered light as a function of time and provides particle size information in terms of hydrodynamic diameter. DLS is a sensitive, non-intrusive, and powerful analytical tool, routinely employed for characterization of macromolecules, colloids and nanoparticles in solution.

The hydrodynamic size (radius) distribution of complexes disclosed herein was measured by DLS. Complexes were prepared as described in Example 1 below at different N/O ratios (e.g., 0.25-3), and diluted to obtain a final HA concentration of 100 mM in a volume of 260 μl DDW. Spectra were collected using CGS-3 (ALV, Langen, Germany) goniometer at a laser power of 20 mW in the He—Ne laser line (632.8 nm). Auto-correlation functions (correlograms) were calculated by ALV/LSE 5003 correlator for a time window of 30 s (a total of 10 times), at an angle of 90 degrees and a temperature of 25° C. The auto-correlation functions were fitted using the CONTIN program (Provencher, Since Direct, 1982, 27: 229-242).

(c) NanoSight

NanoSight analysis is a technique capable of sizing and quantifying nanoparticles through the use of light scattering. Unlike traditional DLS, a charge-coupled device (CCD) camera is used to track the movement of individual nanoparticles in real time, and the system derives their hydrodynamic radius through the Stokes-Einstein equation. NanoSight also goes beyond DLS in that it explicitly quantifies particles below 1 micron and can more accurately characterize polydisperse samples. NanoSight nanoparticle analysis instruments generate videos of a population of nanoparticles moving under Brownian motion in a liquid when illuminated by laser light. Within a specially designed and constructed laser illumination device mounted under a microscope objective, particles in the liquid sample which pass through the beam path are seen by the instrument as small points of light moving rapidly under Brownian motion. This ability of the NanoSight system affords a dynamic analysis of the paths the particles take under Brownian motion over a suitable period of time (e.g., 30 seconds).

The diameter of Q-starch/HA complexes in water solution was determined using the NanoSight technique. Complexes were prepared as described in Example 1 herein at N/O molar ratios of 0.25-3 and diluted to a final concentration of 13 mM HA at a final volume of 2 ml DDW. NanoSight range NS300 instrument (Malvern Instruments, Malvern, UK) equipped with a 642 nm laser module and 650 nm long pass filter was used. All measurements were performed at room temperature in a flow cell (software: NTA 3.1(iss2)). All samples were analyzed under 20× objective and 60 sec video clips were taken.

(d) Cryogenic Transmitting Electron Microscopy (Cryo-TEM)

Cryogenic Transmitting Electron Microscopy (Cryo-TEM), also known as cryo-EM, is a form of cryogenic electron microscopy, more specifically a type of transmission electron microscopy (TEM) where the sample is studied at cryogenic temperatures. The cryogenic temperature range is defined as from −150° C. (−238° F.) to absolute zero (−273° C. or −460° F.), the temperature at which molecular motion comes as close as theoretically possible to ceasing completely, and materials at cryogenic temperatures are as close to a static and highly ordered state as possible. At these extreme conditions, such properties of materials as strength, thermal conductivity, ductility, and electrical resistance are altered.

In transmission electron microscopy, accelerated electrons pass through and interact with the specimen. The interference between scattered and non-scattered electrons results in the so-called phase contrast and image formation. Because electron microscopes require high vacuum, living cells or more generally hydrated samples cannot be examined by this method at room temperature. In cryo-TEM, this problem is solved by embedding the samples in amorphous ice through plunge-freezing in liquid ethane. When imaged at cryogenic temperatures (e.g., −178° C.), the vapor pressure of the so-called vitrified sample is low and the samples can therefore be imaged in their hydrated state. The utility of transmission electron cryomicroscopy stems from the fact that it allows the observation of specimens that have not been stained or fixed in any way, showing them in their native environment. Cryo-TEM provides the determination of macromolecular structures at near-atomic resolution.

The size and geometry in solution of HA, Q-starch and Q-starch-HA complexes were visualized and characterized via direct imaging of the aqueous solution using cryo-TEM. Complexes were prepared as described in Example 1 at N/O of 0.25, at a final HA concentration of 260 mM at 40 μl DDW. A drop of 2.5 μl of the solution was placed on a carbon lacey film supported on a 300 mesh Cu grid (PELCO® TEM, Ted Pella Ltd). Excess liquid was blotted, and the specimen was vitrified via a rapid plunging into liquid ethane precooled with liquid nitrogen in a controlled environment automatic vitrification system (Leica EM GP) where the temperature and the relative humidity are controlled. The samples were examined at −178° C. using FEI Tecnai™ G² 12 TWIN transmission electron microscope operating at 120 kV and equipped with a Gatan 626 Cold Stage Control Unit. The 2D images were taken with Gatan 794 MultiScan charge-coupled device (CCD) camera.

### (v) Skin Treatment

For in vitro experiments, full-thickness skin from porcine ears (from the backside of the ear) was used. The skin was separated from the ear using a surgical scalpel, cut into small pieces of 2×2 cm and stored frozen (−20° C.) until use. Before each experiment, the skin sample was thawed to room temperature for 10 minutes.

### (vi) Skin Electrical Conductivity Measurements

Skin integrity and the effect of ultrasound pre-treatment were evaluated by skin conductivity measurement. Ag/AgCl 4 mm disc electrodes were introduced in both diffusion cell compartments in in vitro experiments. A voltage of 200 mV AC at 10 Hz was applied in vitro using a function generator (Agilent 33120A, Palo Alto, Calif., USA). The current was measured with a Multimeter (Fluke 45 display multimeter, Everett, Wash., USA).

(vii) In Vitro Skin Permeability Measurements Method and Apparatus

Skin permeability measurements were performed in vertical, static glass diffusion cells composed of donor and receiver compartments. The skin was placed between the two separate compartments, with the stratum corneum (SC) facing the donor compartment. The donor compartment was filled with 6 ml of 1% sodium lauryl sulfate (SLS; a commonly used surfactant for increasing efficacy of topically applied formulation) in PBS. For skin samples treated with ultrasound, the ultrasound (QSonica Q700 Sonicator, frequency=20 kHz, 8.2 W/cm² (3% amplitude), probe diameter of 1.3 cm) was applied as a pre-treatment: ultrasound probe was positioned in the donor compartment, at a distance of 8 mm above the skin surface. To minimize thermal effects, a 50% duty cycle mode was chosen (i.e., 1 second on, 1 second off), and the content of the donor compartment was replaced with fresh medium room temperature every 30 seconds (these ultrasound parameters were used since they were found to be optimal in a previous study). To evaluate permeability of the skin, conductivity measurements were conducted at the beginning of the experiment, before and during US exposure. Skin with conductivity higher than 0.7 (kΩ*cm²)⁻¹ was considered to be defective and not used. The US was turned off after 5 min of exposure and during this time, all skin samples reached a conductivity 50-60 fold higher than the initial conductivity.

After US pretreatment, the skin was removed from the diffusion cell, washed with PBS, returned to the cell and 700 μl of fluorescently-labeled HA (HA^{Hylite Fluor 6}) or 700 μl Q-starch-HA complexes wherein either HA is labeled (Q-starch-HA^{Hylite Fluor 6}) or both the Q-starch and HA are labeled (Q-starch^{5-DTAF}-HA^{Hylite Fluor 647}), at a desired N/O molar ratio (e.g., 0.25), and at HA concentration of 260 mM, were placed on the skin for 24 h, after which time, the skin sample were fixed in 4% paraformaldehyde and embedded in paraffin. After deparaffinization, 5 μm thick sections from each sample were cut, placed on slides and stained for histological analysis. The slides were re-hydrated and visualized using a confocal laser scanning microscope. Confocal fluorescence images were acquired on the LSM-880 with Airyscan confocal system from ZEISS (Germany), with plan-apochromat 20×/0.8 DIC M27 objective.

To visualize skin autofluorescence, excitation was done by 488 nm Argon laser and emission was detected in the rage of 490 nm-597 nm. To visualize Hylite™ Fluor 647 labeled HA, excitation was done with 633 nm HeNe laser and emission was detected in rage 638 nm-759 nm.

(viii) Histological Analysis (DAPI Staining)

DAPI (4′,6-diamidino-2-phenylindole) is a blue-fluorescent DNA stain that exhibits ˜20-fold enhancement of fluorescence upon binding to AT regions of dsDNA. It is excited by the violet (405 nm) laser line. As DAPI can pass through an intact cell membrane, it can be used to stain both live and fixed cells. For DAPI staining, skin tissues were fixed with 4% formalin, embedded in paraffin wax. After deparaffinization, 5 μm thick sections were performed using Microtome (Leica RM2255 microtome, England) and rehydrated. Before staining, slides were washed first with xylene (twice, 10 min each), then in 100% ethanol (twice, 10 min each), 95% ethanol (5 min), 70% ethanol (5 min), 50% ethanol (5 min), distilled water (5 min), and PBS (twice, 10 min). After washes, the slides were mounted with DAPI for nucleus visualization.

### Example 1

### Q-Starch-HA Complex Formation and Characterization

Q-starch-HA complexes were prepared at different molar ratios between positively charged amine groups on Q-starch (N), and negatively charged carboxylic groups on HA backbone (O) (N/O molar ratios). The amount of HA (X mg of HA) was predetermined in each measurement, and the amount of carboxylic groups O (mole O) on HA was calculated according to Equations 2:

$$O ( mol 0 ) = \frac{X ( mg of HA )}{Mw of one monomer ( HA ) ( 379 \frac{mg}{m mol} )}$$

The quantity of Q-starch needed for a desired N/O was calculated using Equation 3:

$$N ( gr of Q - starch ) = ( \frac{N ( mol N )}{O ( mol O )} ) \times O ( mol O ) \times 14 ( \frac{g N}{mol N} ) \times \frac{100}{\% N ( \frac{g N}{g Q - starch} )}$$

For Q-starch-HA complexes preparation, stock solutions of Q-starch (or labeled Q-starch), and HA (or labeled HA) were first prepared. For example, 3 mg of Q-starch were dissolved in 7.5 ml DDW to a concentration of 0.4 mg/ml, and 1 mg of HA was dissolved in 1 ml DDW to a concentration of 0.1 mg/ml. The amount of Q-starch (or Q-starch-5-DTAF) for a specific desired N/O ratio was calculated based on the nitrogen content (weight % of N) using Equation 3. The desired amounts of Q-starch solution and HA solution were taken to create the complexes and following gentle vortexing, the samples were incubated at room temperature for 40 min to allow complex formation through self-assembly. Q-starch-HA complexes were prepared in Eppendorf flask such that the amount of HA was kept fixed while the amount of added carrier (Q-starch) was varied to obtain the desired N/O ratio. The HA concentration in each Eppendorf flask containing the complex solution at a final volume of 700 μl was 260 mM HA.

The size, surface charge and morphology of Q-starch/HA complexes are very important parameters since their values will influence their penetration into the skin. Physical characterizations of the Q-starch-HA complexes at different N/O ratios were obtained using Zeta potential for measuring the surface charge, NanoSight for the diameter size, Dynamic Light Scattering (DLS) for hydrodynamic size measurement, and cryogenic Transmitting Electron Microscopy (cryo-TEM) for size and geometry measurement of the complexes, as described in Materials and Methods.

### (i) Complexes Characterization by Dynamic Light Scattering (DLS) and NanoSight

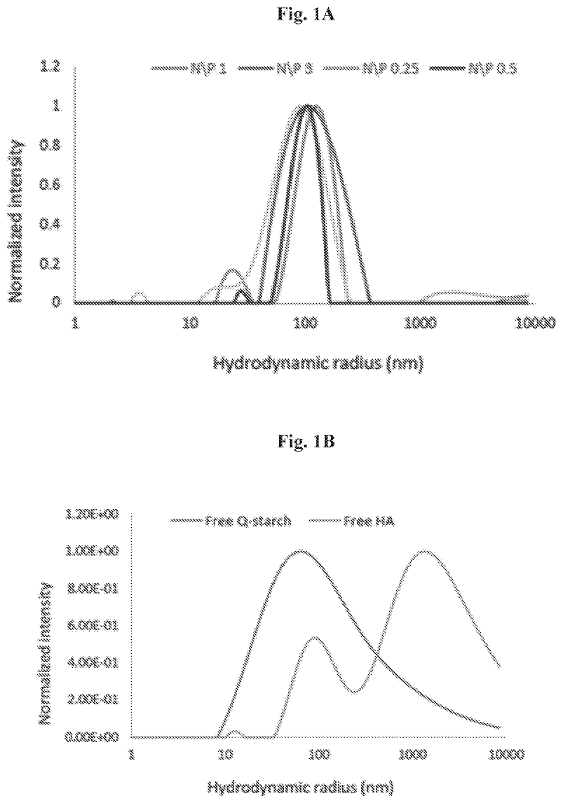

In order to determine the averaged hydrodynamic radius and the diameter of the Q-starch/HA complexes, Dynamic Light Scattering (DLS) and NanoSight techniques were used. Dynamic light scattering was utilized for measuring the scattered light once it interacted with the complexes that were suspended in medium, and for calculating their diffusion coefficient. Their hydrodynamic radius was then calculated by the Stokes-Einstein equation. The size distribution of Q-starch-HA complexes at increasing N/O ratios is shown in FIG. 1A. As seen, the averaged hydrodynamic radius of all N/O molar ratios evaluated (0.25-3) was about 100 nm with no significant difference. In addition, the size distribution of the complexes for each N/O ratio was relatively uniform in size, and the peaks were all within the same range. However, DLS measurements of free HA and free Q-starch demonstrated very wide ranges of hydrodynamic radius sizes, as shown in FIG. 1B. These size distributions clearly demonstrate no evidence of self-internal interactions to form particles, as opposed to the complexes, which demonstrated narrow size distributions.

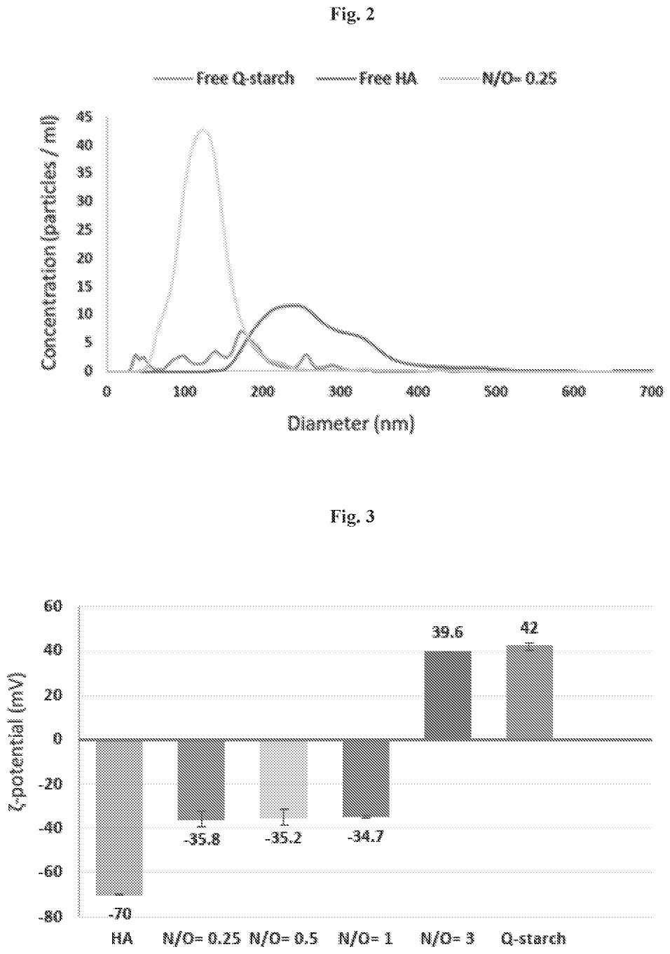

To verify the DLS results, the size diameter of Q-starch/HA complexes was evaluated by another method, NanoSight. FIG. 2 presents representative results of free HA, free Q-starch, and Q-starch/HA complexes having N/O 0.25. It can be seen that both free HA and free Q-starch plots demonstrated a very wide range of size distribution with many unclear peaks, while the complex plot demonstrated relatively narrow size distribution. No significant differences in size distribution (averaged diameters) of Q-starch/HA complexes having increasing N/O ratios, as measured with NanoSight, was observed (results not shown), which is consistent with the results obtained from DLS measurements.

### (ii) Complexes Characterization by Zeta Potential

Zeta potential is a function of the surface charge of a particle, any adsorbed layer at the interface, and the nature and composition of the surrounding suspension medium. In order to determine the complexes surface charge at different N/O molar ratios, ζ-potential measurements were conducted for Q-starch-HA complexes having different N/O ratios, as well as for free HA, and freshly prepared, non-complexed Q-starch. The results are presented in FIG. 3. Negative values were obtained for ζ-potential of non-complexed HA (−70 mV), as expected, due to the negatively charged carboxylic groups of this polymer, and a highly positive ζ-potential value of 42 mV was obtained for non-complexed Q-starch, confirming the presence of the positively charged quaternary amine groups. As shown in FIG. 3, increasing N/O ratios resulted in increasing ζ-potential values from a negative values of ˜−36 mV for N/O 0.25 to a positive value of ˜40 mV) for N/O 3.

(iii) Complexes Characterization by Cryo-Transmitting Electron Microscopy (Cryo-TEM)

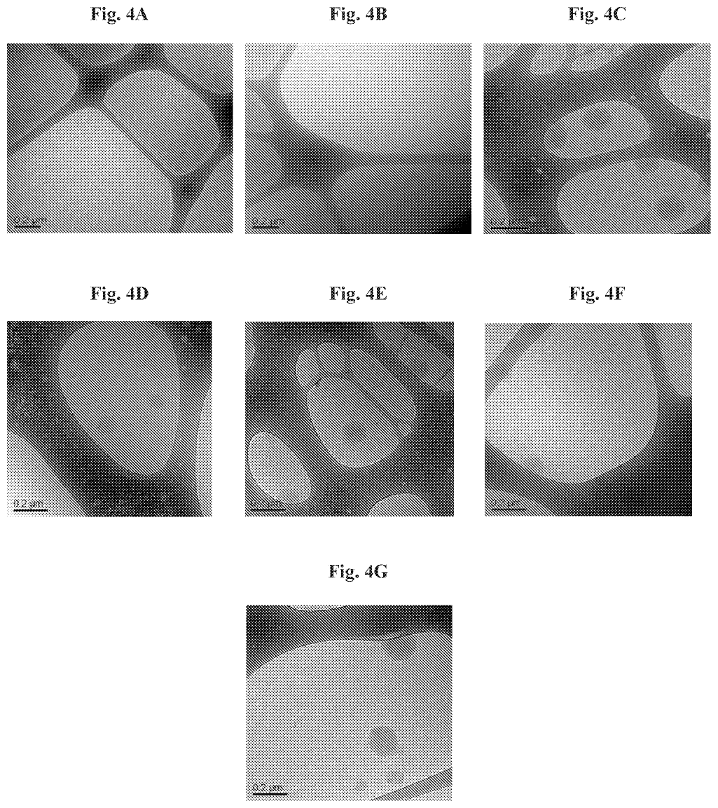

Determination of macromolecular structures or geometric shape of free HA, free Q-starch and Q-starch-HA complex at different N/O (HA and Q-starch were at the same amounts as in the Q-starch-HA complex), was done using Cryo-TEM technology as described in Materials and Methods, and the results are shown in FIGS. 4A-4G. As shown in FIG. 4A, free Q-starch samples could not be clearly visualized by cryo-TEM and only clean grids were observed. This can be explained by the fact that a polymer in aqueous solution is well dissolved, and since the electron density is low, the spread atoms separately can't be seen. Same results were observed also for free HA (FIG. 4B). In contrast, cryo-TEM images of Q-starch/HA complexes (FIGS. 4C-4G) demonstrated mostly small globular and condensed aggregates.

Stability of the Q-starch-HA complexes in an aqueous solution over time was evaluated by assessing their hydrodynamic size 3 h, 24 h and 48 h after they were formed. For example, complexes featuring N/O 0.25 were assessed by Nano Sight and by cryo-TEM, and although their size slightly increased over time (probably due to HA swelling in aqueous medium), their diameter was not doubled or significantly increased, clearly indicating that no aggregates of the complexes were formed, namely, the complexes were stable (results not shown).

### Example 2

### The Effect of Ultrasound Application on Skin Permeability of Hyaluronic Acid Solution

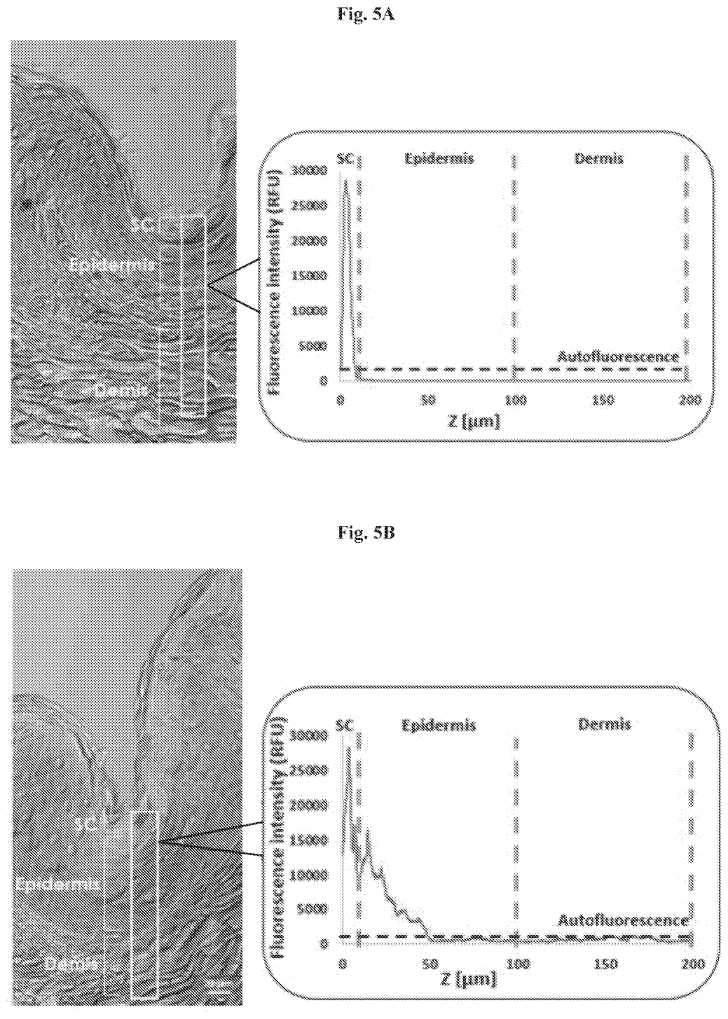

In vitro uptake of a solution of fluorescently labeled high molecular weight (HMW) HA by porcine skin samples was measured with or without preapplication of low frequency ultrasound to the skin samples, using diffusion cell method and apparatus described in Materials and Methods. Prior to each permeability measurement, 0.3% (w/v) HA solution was prepared by dissolving 3.6 mg HA (Mw 1500 KDa) in 1.2 ml DDW in a glass vial and stirring at room temperature until full dissolution. The HA was fluorescently labeled with Hylite™ Fluor 647 amine dye (herein designated HA^{Hylite Fluor 647}) as described in Materials and Methods. Before labeled HA was provided to the skin samples, 5 min US application (20 KHz, 8.2 W/cm², Duty Cycle=50%) was performed as a pretreatment as described in Materials and Methods. Confocal microscope images of an exemplary porcine ear skin sections 24 h after US application are shown in FIGS. 5A-5C. Fluorescence intensities of labeled HA as a function of distance from the SC down to a depth of 200 μm, calculated for pixels of an exemplary rectangular cross-layers section by Image j is presented in respective graphs.

As shown in FIG. 5A, in absence of US pretreatment, all fluorescence confined to the SC layer, clearly indicating that HA did not penetrate the skin. On the other hand, in skin samples that were pretreated with US, fluorescence intensity was higher in the epidermis, scattered in layers below the SC layer, up to a depth of 50 μm (FIG. 5B). These results confirm that US application can affect skin penetration beyond the SC layer.

One possible explanation for this phenomenon of SC permeability enhancement is the mechanical effect caused by US application, such as cavitation. As discussed herein, both microjets and shockwaves may be responsible for enhanced SC permeability.

Yet, HA barely penetrated deeper to the dermis, which is the target layer for HA biological activity. This can be explained by the fact that the HMW HA is a large molecule, especially in aqueous solution, which slows down its diffusion through the skin.

### Example 3

### The Effect of Complexing Hyaluronic Acid with Quaternary Starch on its Skin Permeability

In order to facilitate HA penetration deep into the dermis, HA hydrodynamic size was condensed, and its radius was reduced to a penetrable size by complexing the negatively charged HA with a cationic carrier via self-assembly. Complexing HA with a carrier has the additional benefits of extending HA half-life, thus, providing HA with longer stability and retention time in the skin. In this study, positively charged modified starch (Q-starch) was used as HA carrier.

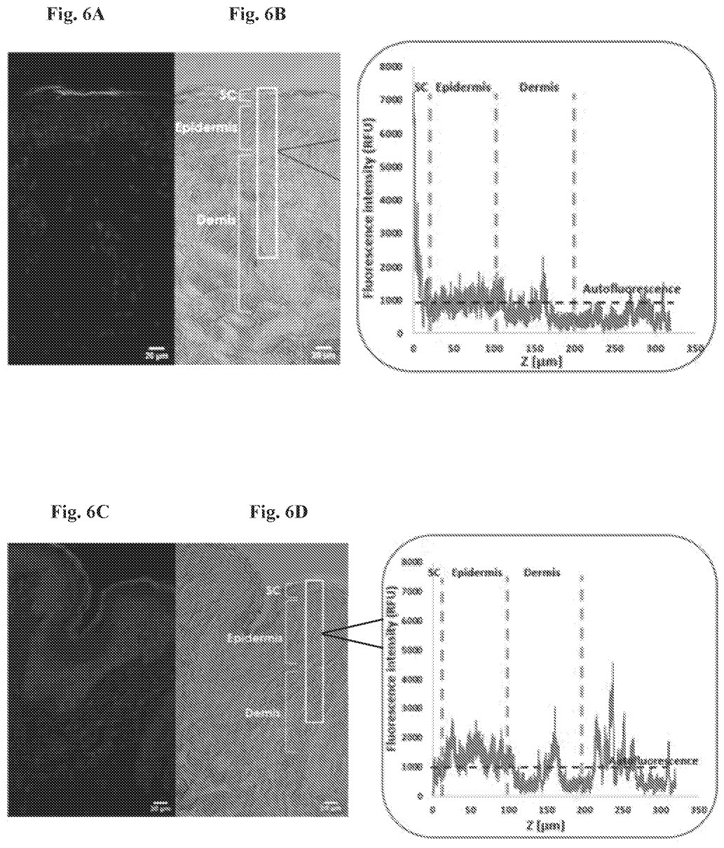

Entry of Q-starch-HA complexes to deep skin layers following their topical application on porcine ear skin samples was studied in vitro using the diffusion cells method and apparatus described in Materials and Methods. HA was labeled with Hylite™ Fluor 647 amine dye (HA^{Hylite Fluor 647}) and the complex, Q-starch-HA^{Hylite Fluor 647} was formed with N/O molar ratio of 0.25, and topically applied on the skin samples for a period of 24 hours in absence or presence of ultrasound pre-application (for 5 min) to the skin. As a control group, untreated (no US application and no complex administration) ear skin sections were used. This control group was used for assessing the autofluorescence at various depths or layers of the skin samples Visualization of histologically stained treated and non-treated (control) porcine skin cross-sections was done with confocal microscope (excitation was done with 633 nm HeNe laser and emission was detected in rage 638 nm-759 nm), and exemplary confocal and bright field images of the treated skin are presented in FIGS. 6A-6D. In the DAPI stained cross-sections, complexed HA^{Hylite Fluor 647} appeared as red staining and cells nuclei were stained blue. In the bright field confocal images, complexed HA^{Hylite Fluor 647} appeared pinkish-red. Fluorescence intensity of the Q-starch-HA^{Hylite Fluor 647} complexes as a function of distance from SC down to a depth of 350 μm calculated (by Image j) per pixel of an arbitrary skin cross-layers section indicated by a rectangle in the images, is presented for both US pre-treated and non-treated skins. For convenience, herein, fluorescence intensity calculated for an arbitrary pixel, is referred to as “pixel fluorescence intensity”.

As seen in FIGS. 6A and 6B, in skin which was not pre-treated with US before complexed HA application, the topically administered complexes mostly remained at the top layer of the skin, the SC layer. The pixel fluorescence intensity, as calculated, was higher in the SC layer than the epidermis and dermis layers (although some fluorescence was detected in these deeper layers as well). In contrast, in skin samples that were pretreated for 5 min with US before topically applying the Q-starch-HA^{Hylite Fluor 647} complex for 24 h, the complex penetrated through the SC barrier into the epidermis, including the basal cell layer in the dermis (FIGS. 6C-6D). As seen in FIG. 6D, the pixel fluorescence intensity was higher in the epidermis and dermis layers than in the SC layer.

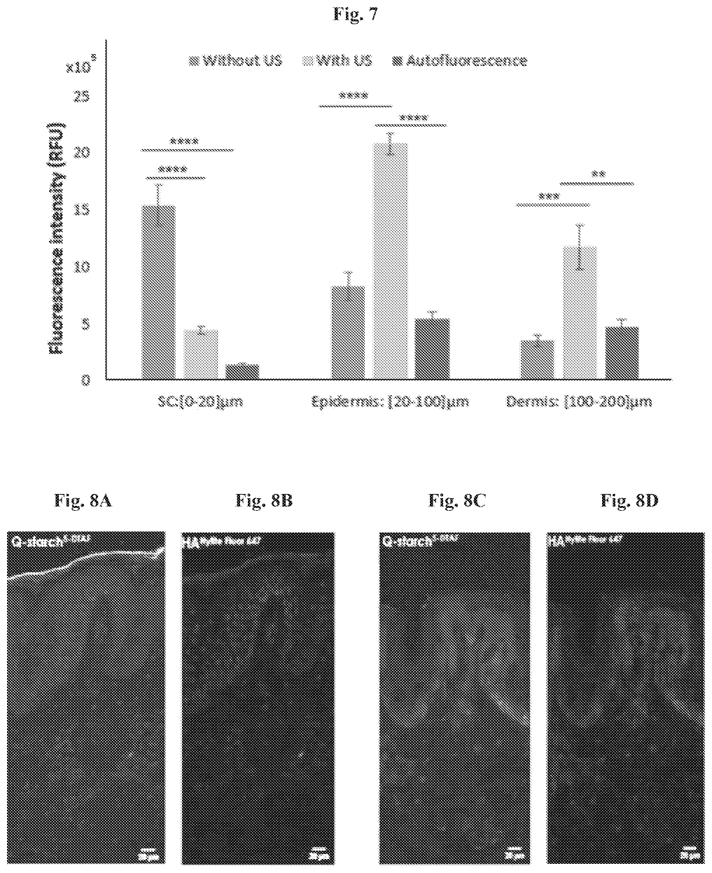

In order to quantify the difference in fluorescence intensity of Q-starch/HA complexes in deep skin layers in the US pre-treated versus none US pre-treated skin groups, three groups of skin samples were observed: (i) skin samples topically applied with labeled Q-Sratch-HA complex (Q-starch-HA^{Hylite Fluor 647}) for 24 hours; (ii) skin samples treated with ultrasound for 5 min and then topically applied with Q-starch-HA^{Hylite Fluor 647} for 24 hours; and (iii) control group—skin samples not treated with neither ultrasound nor labeled complex. For each skin sample, three randomly cross-layers rectangular sections were selected, and pixel fluorescence intensity thereof was calculated. The results are presented in FIG. 7.

As shown in FIG. 7, skin that was pretreated with US demonstrated higher fluorescence intensity in the epidermis and dermis than in the SC layer as compared to skin that was not pre-treated by US application.

As further shown in FIG. 7, in the SC layer (0-20 μm), autofluorescence as measured in the control group was significantly lower than the HA^{Hylite Fluor 647} fluorescence measured in both US pre-treated and none pre-treated skins. However, in the epidermis (20-100 μm) the difference in fluorescence intensity between skins that were not pre-treated with US and autofluorescence was dramatically smaller, and in the dermis (100-2000 μm), autofluorescence was even higher than HA^{Hylite Fluor 647} fluorescence. On the other hand, in skin that was pretreated with US, HA^{Hylite Fluor 647} fluorescence intensity was significantly higher than the autofluorescence in both the epidermis and dermis, clearly indicating that the complexes indeed penetrated to these layers, as opposed to skin that was not pretreated with US.

From these results, it can be concluded that a combination of ultrasound pre-application and a carrier for delivering HA resulted in highly effective penetration of HA to deeper layers of the skin, including the target layer (dermis).

### Example 4

### Q-Starch/HA Complexes Stability in Skin Layers

In order to evaluate stability of Q-starch/HA complexes in skin layers under the treatment conditions described in Example 3 above, the carrier Q-starch was labeled with 5-(4,6-dichlorotriazinyl) aminofluorescein (5-DTAF) (green) and HA was labeled with Hylite™ Fluor 647 (red), and a complex Q-starch^{5-DTAF}-HA^{Hylite Fluor 647} was formed as described in Materials and Methods, having N/O of 0.25 and final a concentration of 260 mM of complexed HA. Fixed samples were stained with DAPI to assess intact, nucleated cells of skin layers beneath the SC (the SC comprises dead, unnucleated cells). Stained sample were sectioned and visualized using a confocal microscope.

Confocal images of porcine skin samples were taken 24 hours after topical administrations of labeled complexes either following a preceding 5-min US application or in absence of prior US application. Exemplary confocal images are presented in FIGS. 8A-8D.

As seen in FIGS. 8A-8B, in absence of US pre-treatment, topical administration of Q-starch^{5-DTAF}-HA^{Hylite Fluor 647} for 24 h resulted in both green and red staining residing in the SC in similar patterns. Penetration of the labeled complex into the epidermis in skin samples pre-treated with US application is shown as green staining due to Q-starch^{5-DTAF} presence and red staining due to HA^{Hylite Fluor} 647 presence, wherein the green and red staining patterns are similar (FIGS. 8C-8D). These results indicate that HA did not dis-assembled from Q-starch, and the complex retained its stability in deep skin layers.

### Example 5

### In Vivo Permeability Study

In order to evaluate, in vivo, skin uptake of Q-starch-hyaluronic acid complexes in mice, the following study is designed.

Mice are divided into the following groups: Group I: control—mice not subjected to any treatment; Group II: mice treated with Q-starch-HA without US pre-treatment; and Group III: mice subjected to US pre-treatment followed by administration of Q-starch-HA complexes (in different N/O molar ratios). Hyaluronic acid is labeled with Hylite™ Fluor 647 amine dye (HA^{Hylite Fluor 647}) and contacted with Q-starch to form Q-starch-HA^{Hylite Fluor 647} complex. The following step in the study protocol are applied:

1. The back of each mice is shaved using haircut clippers. Then, mice are anesthetized with isoflurane.

2. A rubber ring with a plastic cylinder is attached on top of the shaved skin using biological glue, and 2 ml of PBS is poured inside the chamber.

3. About 1 cm cut near the tail is made. One conductivity electrode is positioned inside the chamber, and another inside the incision, for measurement of the initial conductivity of the skin.

4. Afterward, the PBS in the chamber is replaced with 1% SLS in PBS and skin conductivity is measured again.

5. Mice in Group III are subjected to US application (QSonica Q700 Sonicator, frequency=20 kHz, 6.1-10.5 W/cm², probe diameter of 1.3 cm), conducted as a pre-treatment: the ultrasound probe is positioned in the plastic cylinder, 8 mm from the surface of the skin. To minimize thermal effects, a 50% duty cycle mode is chosen (i.e., 0.5 second on, 0.5 second off), and the content of the plastic cylinder is replaced with fresh medium every 20-30 seconds (depending on the temperature). To evaluate the permeability of the skin, conductivity measurements are conducted during ultrasound exposure. Ultrasound application is turned off when the conductivity reaches 50-70-fold of the initial conductivity or a predetermined value of 0.70 (kΩ*cm²)⁻¹.

6. for mice in group II and III, the plastic cylinder is removed, and Q-starch-HA^{Hylite Fluor 647} complexes prepared 40 minutes before US application are placed inside the rubber ring. Parafilm cover is applied to prevent fluid leakage from the ring.

7. Twenty our hours after complex administrations, mice in Groups I-III are sacrificed, and the skin is removed, fixed in 4% formalin and cut to 5 μm slices (slides).

8. The slides are rehydrated and visualize using a confocal laser scanning microscope.

### Example 6

### In Vivo Model for UV Radiation Induced Wrinkles

To create skin aging model in mice, there is a need to cause collagen degradation in the dermis layer and therefore, as a first step, this degradation is induced by ultraviolet (UV) radiation conducted for a period of about 5-12 weeks, until wrinkles become apparent and collagen fibers decreases.

Epidermal and dermal thickness is evaluated by light microscope, elasticity of the skin and skin hydration is measured by different devices with the reasonable expectation that the elasticity and skin hydration will decrease. The effect of Q-starch-HA^{Hylite Fluor 647} complexes application on mice skin appearance with or without US pre-treatment is analyzed and examined through histological staining. For histological analysis, the skin samples need to maintain their structure and function as they were in the animal body, therefore the sample undergoes several stages: fixation, embedding, sectioning, and staining. Hematoxylin and Eosin (H&E) staining is conducted to evaluate epidermal and dermal thickness.

1. A non-invasive method for preventing or treating skin aging processes in a subject in need thereof, the method comprising the steps of:
(a) applying ultrasound treatment to a skin surface of the subject for a period of from about 5 sec to about 5 min;
(b) topically administrating to the ultrasound-treated skin surface at least one of: free hyaluronic acid (HA) or HA complexed with a polysaccharide (HA-polysaccharide complex); and
(c) optionally, repeating at least one of step (a) or (b) at least one more time,
thereby non-invasively preventing or treating skin aging processes in the subject.

2. The method of claim 1, wherein the molecular weight of hyaluronic acid is at least one of: lower than 100 kDa, lower than 500 kDa, higher than 500 kDa, or in the range of from 300 kDa to 800 kDa, from 500 kDa to 1500 kDa, from 500 kDa to 8000 kDa, from 1000 kDa to 5000 kDa, or from 500 kDa to 3000 kDa.

3. The method of claim 1, wherein preventing or treating skin aging processes is at least one of: maintaining skin hydration, restoration or improvement of collagen production, delaying, reducing or preventing aging process selected from the group consisting of wrinkling, skin atrophy, or loss of skin elasticity, or reducing aging indicators associated with loss of mechanical properties.

4. (canceled)

5. A non-invasive method for transdermal delivery of one or more glycosaminoglycans (GAGs) in a subject in need thereof, the method comprising the steps of:
(a) optionally, forming a complex comprising one or more GAGs and at least one polysaccharide (GAG-polysaccharide complex);
(b) applying ultrasound treatment to a skin surface of the subject for a period of from about 5 sec to about 5 min;
(c) topically administrating one or more GAGs and/or one or more GAG-polysaccharide complexes to the ultrasound-treated skin surface;
(d) optionally, applying a further ultrasound treatment to the skin surface for a period of from about 5 sec to about 5 min; and
(e) optionally, topically administrating to the ultrasound-treated skin surface, a further amount of one or more GAGs and/or one or more GAG-polysaccharide complexes,
thereby transdermally delivering one or more GAGs in the subject.

6. The method of claim 5, wherein the GAG is at least one of: hyaluronic acid, heparin, heparan sulfate, chondroitin sulfate, dermatan sulfate, or keratan sulfate of a molecular weight of from 500 kDa to 8000 KDa, from 500 kDa to 3000 kDa, or of from 300 kDa to 800 KDa.

7. The method of claim 5, wherein at least one of step (d) or step (e) is conducted 0, 1, 2, 3 or more times, and:
(i) step (a) is applied, and at least one GAG-polysaccharide complex is topically administered in step (c);
(ii) step (a) is applied, and at least one GAG-polysaccharide complex is topically administered in step (e), or
(iii) step (a) is applied, and at least one GAG-polysaccharide complex is topically administered in both steps (c) and (e).

8. (canceled)

9. The method of claim 6, wherein the GAG is hyaluronic acid.

10. (canceled)

11. The method of claim 1, wherein the polysaccharide is a modified or non-modified polysaccharide being at least one of starch, chitosan, pectin, cellulose, dextran, or galactan.

12. The method of claim 11, wherein the polysaccharide is a modified polysaccharide substitution with one or more positively charged chemical moieties.

13. (canceled)

14. The method of claim 12, wherein the polysaccharide is starch substituted with quaternary amine groups

15. The method of claim 14, wherein the starch substituted with quaternary amine groups is complexed with hyaluronic acid, and the molar ratio of positively charged quaternary amine groups on the starch and negatively charged carboxyl groups on hyaluronic acid (N/O molar ratio), is in a range of from about 0.20 to about 3.00, from about 0.22 to about 0.50, form about 0.25 to about 1.50, from about 1.00 to about 2.50, or about 0.25.

16. The method of claim 1, wherein ultrasound is applied for a period of from about 30 sec to about 2 min.

17. A complex of hyaluronic acid and a chemically modified starch.

18. The complex of claim 17, wherein the starch is modified by substitution with one or more positively charged chemical moieties.

19. The complex of claim 18, wherein the positively charged chemical moiety is a quaternary amine group.

20. The complex of claim 18, wherein the molar ratio of positively charged chemical moieties of the modified starch and negatively charged carboxyl groups of hyaluronic acid (N/O molar ratio) is in a range of from about 0.20 to about 3.00 or from about 0.25 to about 1.5.

21. The complex of claim 17, formulated as a cosmetic composition or a therapeutic.

22-25. (canceled)

26. The method of claim 5, wherein ultrasound is applied for a period of from about 30 sec to about 2 min.
